SEQUENCE LISTING (20 sequences)

SEQ ID NO: 1 - DNA, 3709 bp, Homo sapiens
  CDS (170)..(3532)
ggcacgagtctatctcatcaataaacattcactgggttcttggtttaaggggtttgagaagcttgccacctcccagcgtttattattatttttttcttttgccccgacctttagcctctgggccatttgtaatacatatttaatttttagtcacaggaaataagtaatcatggtgctcaacagtttggataagatgattcaactccagaaaaacactgccaacatcaggaatatttgtgttttggctcatgttgaccatggaaaaactactctggctgactgtcttatatctagcaatggaatcatctccagccgcctagcaggcaagttaaggtacatggacagcagagaagatgaacagatccgagggatcactatgaaatccagtgccatttccctacattatgcaacaggtaatgaggagtacctgatcaatctgatagactctccaggacacgtggacttttcctcagaagtatcaaccgctgttcgcatttgtgatggatgcatcattgtggtagatgctgtggaaggagtctgtccacagacacaggcagttctgcgacaagcttggcttgaaaacatccgtccggttttagtgattaataagattgatcgcttgatagtggaactgaaattcaccccacaagaggcctattctcacctcaagaatattttagaacagattaatgcgctcacagggactctttttacttctaaagtcctagaagaaagagcagagagggagactgaatcccaagtgaatccaaattctgaacaaggagagcaagtatatgactggagcactggcttggaggacacagatgattctcacctttacttctctccagaacagggaaatgtggtgtttaccagtgcaatagatgggtggggctttggaattgagcacttcgccagaatctacagtcaaaaaattggcatcaaaaaggaagttcttatgaaaaccttgtggggagattactatataaatatgaaggctaaaaagatcatgaagggtgatcaggccaaaggaaagaaacctttatttgtacagttgatcctggaaaatatatggagtttgtatgatgctgttttgaaaaaggacaaagacaaaattgataaaatagtgacttctttaggattaaaaattggagcccgggaggcacgacattcagaccctaaagttcagatcaacgccatttgcagtcagtggctacccatatcccatgctgttcttgctatggtgtgtcagaaacttcctagtccccttgatattacagctgagagagtggagagactgatgtgcacaggatcacaaacttttgactcttttccaccagaaactcaagcactgaaagcagcttttatgaaatgtggaagtgaggacactgctccagttattatatttgtttccaaaatgtttgcagttgatgctaaggccttgcctcagaataagccaaggcctctcactcaagaagaaattgctcagagacgtgagcgtgcaagacaaaggcatgcagagaagcttgcagcagcacagggacaggcacccttggagcccacccaagatgggagtgccattgaaacatgtccaaaaggagacgagccaagaggtgacgagcaacaggtggaaagtatgacccctaaacctgtgctccaggaagaaaacaaccaagagtcttttattgcatttgctcgggtgttcagtggtgtggctcgaagaggaaagaaaatttttgtcttggggcccaaatacagtcctcttgagtttttacgaagggtaccattaggcttctcagctccaccagatggcctcccccaagtcccccacatggcatactgtgctctggaaaacctgtatcttctgatgggaagggaactggaatatctagaggaggtacctccaggaaatgtgctaggaataggaggccttcaagattttgtgctgaaatctgcaacactgtgtagcctgccatcctgcccaccatttataccactcaacttcgaagccactcctattgtgagagttgctgttgaaccaaaacatccaagtgaaatgcctcagctcgtaaaaggaatgaaactgttaaaccaggctgatccctgtgtccagattttaattcaggaaacgggagagcacgttttagtcacagcaggagaagtccaccttcagcgatgcctggatgacttaaaagaaaggtttgcaaagattcatatcagtgtatctgaacctattattccattcagagaaacaatcacaaaacccccaaaagttgacatggtcaatgaagaaataggcaaacagcaaaaagttgcagtcatacaccaaatgaaagaagatcaaagcaaaatccctgaaggaatccaagttgactctgacgggctaatcaccataacaactcccaataaacttgccacgctcagtgttcgagccatgccccttccagaagaagtcacccagattctggaagaaaatagtgatttgattcgttctatggagcagttgacatcctctttgaatgagggtgaaaatactcacatgattcatcagaagacccaagagaaaatttgggaattcaaaggaaaactggagcaacacctaacagggagaagatggaggaacattgttgaccaaatctggtcatttggcccaagaaaatgtgggcccaacatactagtcaataaaagtgaagattttcagaactcagtatggacaggtccagctgacaaagcttcaaaagaagccagtagataccgagatttgggcaatagcattgtgagtggcttccaactagcaaccctctctggccccatgtgtgaggagcctctcatgggtgtctgttttgttctggaaaaatgggacctaagtaaatttgaggaacaaggagcaagtgatctggcaaaagagggacaggaggaaaatgaaacctgttctggtggaaatgaaaaccaagagctacaagatggctgctctgaggcctttgagaagaggacatcacagaaaggagaatctccactcactgactgctatggacctttctcaggacagctaattgccaccatgaaagaagcatgtcgctatgcactgcaagtgaaacctcagcgcctgatggcagctatgtacacatgtgacatcatggccactggtgatgttctcggtcgagtctatgctgtcttgtcaaagagagaaggtcgggtacttcaagaagaaatgaaagaagggacagacatgttcatcatcaaggctgtgctgcctgttgctgaaagctttggttttgctgatgaaatcaggaagaggacaagtggcctggccagcccacaactagtattcagccattgggagatcattcccagtgaccccttctgggtgccaactactgaggaggaatacttgcactttggggagaaggctgactctgagaaccaagcccggaagtacatgaacgcagtacgaaagcggaaggggctttatgtggaagaaaagattgtggagcatgcagaaaagcagaggacactcagcaaaaataagtagctacctactactggtggattcttttccttatagtgaatttaaaagtatcatcaagggtttaatattgggaaaatttctttttgccacattatctctgtttattcactttcaataaagttgatccatataaatattttaaaaaaaaaaaaaaaaaaaaaaaaaaaaaaaaaaaaaaaa

SEQ ID NO: 2 - protein, 1120 aa, Homo sapiens [sequence omitted]

SEQ ID NO: 3 - DNA, 3533 bp, Homo sapiens
  CDS (14)..(3478)
ggcacgagcggtcatggaggcgggcgccggagccggcgcgggagccgcgggctggagctgcccgggcccaggacccacagtgaccactctaggctcctatgaggcttccgagggctgtgagaggaagaagggccaacgctgggggtccctggaacgacgggggatgcaagctatggagggggaggtgttactcccagctctctatgaggaggaagaggaagaggaagaggaggaagaagaggtggaagaagaagaagaacaagtgcagaaaggtggcagtgttggctctctgtcagtcaacaagcaccggggactgagcctcacggagacagagctggaggagctgcgggctcaggtgctgcagctggtggcagaactggaggagacccgggaactggcagggcagcatgaggatgactccttggagctacaggggctcctggaggatgaacggctagccagcgcccagcaggcagaggtgttcaccaagcagatccagcagctccaaggtgagctgcgttctctacgggaggagatttccctgttagagcatgagaaagaaagcgaacttaaggaaatagaacaggaattgcatttggcccaggctgagatccagagtctgcggcaagcagcagaggattccgcaactgaacatgagagtgacatagcatccctgcaggaggatctctgccggatgcagaatgaacttgaagacatggaacgcattcggggagattatgagatggagatcgcctccctccgtgcagaaatggaaatgaagagctctgaaccatccggtagtttaggtctctcagattactctgggttacaagaagaactgcaggagctgcgggaacgctaccatttcctgaatgaggaataccgggccctgcaggagagcaacagcagcctcacggggcagcttgcagatctggagagtgagaggacacagagagcaacagagagatggctgcagtcccaaacactgagtatgacgtcagcagagtctcagacttcagaaatggatttcttagagcctgatcctgaaatgcagttgttacggcagcagctacgggatgctgaagagcagatgcatggcatgaagaacaagtgtcaggaattgtgttgtgagttggaagagctacagcatcatcgccaggtcagtgaggaggagcagaggcggctgcagagggagctcaagtgtgctcagaatgaggtgcttcggtttcagacctcccacagtgtcacccaggagttactgtgccggctgcagaagctgcacctccagcaccagaacgtcacatgtgagaaggaaaagctgctggaacggcagcagcagctgcaggaggagctgcagtgccatgaggcagagctgcagcacctcagggatacggtggcctccttcaaagagagcaatgagaaggacacagagacgcacgctcagcttcaggagatgaagcagctgtaccaggccagcaaggacgagctggagcggcagaagcacatgtatgaccagctggagcaggacctcctgctctgccagctggagctgaaagagctcaaggcctcccaccccattccggaggacaaaggaaagtgtgctaataagtgtgacacactgctgtccagactgacagaattgcaggaaaagtacaaggccagccagaaggagatggggcagctgcagatggagcagtgtgagctcctggaggatcagaggaggatgcaggaggagcagggccagctgcaggaagagctgcacaggctcacactgccactgccaaagagtggcctcttactcaagagtcaggagctactcaccaagttagaagacctgtgtgagctgcagctgctctaccaaggcatgcaggaggaacagaagaagctgatacagaaccaagactgtgtattaaaagaacaattagagatccacgaagagctgcgacgtttcaaagagtctcatttccaggaagtgttggagaatcccgatgattccaaattggctaagtcctccaaatgtaatcgaaacaagcaatccaagctgctcatggagcagatgcaggccctgcaggtgatgtatgacgccggtcaggcgaagcaggagctcttgcagcaagagcaagggaggctcctagaggagcggaagaggctgcaggcagacttgcagctctgcctggaagaaatgcagctgcttcaagtccagtccccttctataaaaatgagccttgagtcctacgggaagagctatggtagcatggtccccagcaatgagaactgtcgcaagacttatgataccactgtggatgacaatgagagctattacaagagttacaccagcacccagaccagcagcaagagctttctcaagagctatgacagcagcaccagtgccagtgaggcctatgggaagagttactgcactaccagcaacagcagcattacctataagaagagttacggcagcaccagtagctctgacacctgccagaagagttttgtcagcagctgcactgacgaggaacctgctgagcctgaagacatggagcgctttgaggaaatggttgtgaaagtgctgatcaagctgcaggcggtgcaggccatgtaccagataagccaggaggaacacagccagctgcaagagcagatggaaaagttactggccaagcagaaagacctgaaggaagagctggatgcctgtgaaagggagttcaaggagtgcatggaatgccttgaaaagcccatggccccccagaacgacaagaatgagatcaaagaactgcagaccaagctgcgggagctgcagctgcaataccaggctagcatggatgagcaggggcagcttctggtagtgcaggagcagctggaggggcagctgcagtgctgccaggaggagctccgccagctcagggagaagaggccttctgttgtcaaagaagcccgggggaagaatgctaataagaacatgaacaagaatgccaatggggttaaaatgaaaaaggtgaccaagccatgctcggatacttctgagagcgaccttgagaccagaaagaagatcaggaggaaaatgaagaggacaaagaggaagaggagaaggaagaagacagtgaagaggaggaagatgacgccgactcttcccttgaaagtcccgaagaaaataaccccctcagactttccgagagcaaaaagaacatgtttgggttgtggaagcctatggtattcttggctattgcagctgtggctctgtatgtgttacccaacatgcgacagcaggagtcagagttctgcctcatggagtgatggcagaccttggccagcgcgagggcagatccccagtggccaccaccctcagctttgggcaggacacactgtgccagaaccctccccatatgttccatgtgtccccatctcctcagcctcagtcacccaggctgaaaaggcttgtggggagcggctgacttccatctcctgccttgtgtaagaacctgagttccttgtaattaaatatcaactgaattaaaaaaaaaaaaaaaaaa

SEQ ID NO: 4 - protein, 1154 aa, Homo sapiens
MEAGAGAGAGAAGWSCPGPGPTVTTLGSYEASEGCERKKGQRWGSLERRGMQAMEGEVLLPALYEEEEEEEEEEEEVEEEEEQVQKGGSVGSLSVNKHRGLSLTETELEELRAQVLQLVAELEETRELAGQHEDDSLELQGLLEDERLASAQQAEVFTKQIQQLQGELRSLREEISLLEHEKESELKEIEQELHLAQAEIQSLRQAAEDSATEHESDIASLQEDLCRMQNELEDMERIRGDYEMEIASLRAEMEMKSSEPSGSLGLSDYSGLQEELQELRERYHFLNEEYRALQESNSSLTGQLADLESERTQRATERWLQSQTLSMTSAESQTSEMDFLEPDPEMQLLRQQLRDAEEQMHGMKNKCQELCCELEELQHHRQVSEEEQRRLQRELKCAQNEVLRFQTSHSVTQELLCRLQKLHLQHQNVTCEKEKLLERQQQLQEELQCHEAELQHLRDTVASFKESNEKDTETHAQLQEMKQLYQASKDELERQKHMYDQLEQDLLLCQLELKELKASHPIPEDKGKCANKCDTLLSRLTELQEKYKASQKEMGQLQMEQCELLEDQRRMQEEQGQLQEELHRLTLPLPKSGLLLKSQELLTKLEDLCELQLLYQGMQEEQKKLIQNQDCVLKEQLEIHEELRRFKESHFQEVLENPDDSKLAKSSKCNRNKQSKLLMEQMQALQVMYDAGQAKQELLQQEQGRLLEERKRLQADLQLCLEEMQLLQVQSPSIKMSLESYGKSYGSMVPSNENCRKTYDTTVDDNESYYKSYTSTQTSSKSFLKSYDSSTSASEAYGKSYCTTSNSSITYKKSYGSTSSSDTCQKSFVSSCTDEEPAEPEDMERFEEMVVKVLIKLQAVQAMYQISQEEHSQLQEQMEKLLAKQKDLKEELDACEREFKECMECLEKPMAPQNDKNEIKELQTKLRELQLQYQASMDEQGQLLVVQEQLEGQLQCCQEELRQLREKRPSVVKEARGKNANKNMNKNANGVKMKKVTKPCSDTSESDLETRKKIRRKMKRTKRKRRRKKTVKRRKMTPTLPLKVPKKITPSDFPRAKRTCLGCGSLWYSWLLQLWLCMCYPTCDSRSQSSASWSDGRPWPARGQIPSGHHPQLWAGHTVPEPSPYVPCVPISSASVTQAEKACGERLTSISCLV

SEQ ID NO: 5 - DNA, 3714 bp, Homo sapiens
  CDS (14)..(3478)
ggcacgagcggtcatggaggcgggcgccggagccggcgcgggagccgcgggctggagctgcccgggcccaggacccacagtgaccactctaggctcctatgaggcttccgagggctgtgagaggaagaagggccaacgctgggggtccctggaacgacgggggatgcaagctatggagggggaggtgttactcccagctctctatgaggaggaagaggaagaggaagaggaggaagaagaggtggaagaagaagaagaacaagtgcagaaaggtggcagtgttggctctctgtcagtcaacaagcaccggggactgagcctcacggagacagagctggaggagctgcgggctcaggtgctgcagctggtggcagaactggaggagacccgggaactggcagggcagcatgaggatgactccttggagctacaggggctcctggaggatgaacggctagccagcgcccagcaggcagaggtgttcaccaagcagatccagcagctccaaggtgagctgcgttctctacgggaggagatttccctgttagagcatgagaaagaaagcgaacttaaggaaatagaacaggaattgcatttggcccaggctgagatccagagtctgcggcaagcagcagaggattccgcaactgaacatgagagtgacatagcatccctgcaggaggatctctgccggatgcagaatgaacttgaagacatggaacgcattcggggagattatgagatggagatcgcctccctccgtgcagaaatggaaatgaagagctctgaaccatccggtagtttaggtctctcagattactctgggttacaagaagaactgcaggagctgcgggaacgctaccatttcctgaatgaggaataccgggccctgcaggagagcaacagcagcctcacggggcagcttgcagatctggagagtgagaggacacagagagcaacagagagatggctgcagtcccaaacactgagtatgacgtcagcagagtctcagacttcagaaatggatttcttagagcctgatcctgaaatgcagttgttacggcagcagctacgggatgctgaagagcagatgcatggcatgaagaacaagtgtcaggaattgtgttgtgagttggaagagctacagcatcatcgccaggtcagtgaggaggagcagaggcggctgcagagggagctcaagtgtgctcagaatgaggtgcttcggtttcagacctcccacagtgtcacccaggagttactgtgccggctgcagaagctgcacctccagcaccagaacgtcacatgtgagaaggaaaagctgctggaacggcagcagcagctgcaggaggagctgcagtgccatgaggcagagctgcagcacctcagggatacggtggcctccttcaaagagagcaatgagaaggacacagagacgcacgctcagcttcaggagatgaagcagctgtaccaggccagcaaggacgagctggagcggcagaagcacatgtatgaccagctggagcaggacctcctgctctgccagctggagctgaaagagctcaaggcctcccaccccattccggaggacaaaggaaagtgtgctaataagtgtgacacactgctgtccagactgacagaattgcaggaaaagtacaaggccagccagaaggagatggggcagctgcagatggagcagtgtgagctcctggaggatcagaggaggatgcaggaggagcagggccagctgcaggaagagctgcacaggctcacactgccactgccaaagagtggcctcttactcaagagtcaggagctactcaccaagttagaagacctgtgtgagctgcagctgctctaccaaggcatgcaggaggaacagaagaagctgatacagaaccaagactgtgtattaaaagaacaattagagatccacgaagagctgcgacgtttcaaagagtctcatttccaggaagtgttggagaatcccgatgattccaaattggctaagtcctccaaatgtaatcgaaacaagcaatccaagctgctcatggagcagatgcaggccctgcaggtgatgtatgacgccggtcaggcgaagcaggagctcttgcagcaagagcaagggaggctcctagaggagcggaagaggctgcaggcagacttgcagctctgcctggaagaaatgcagctgcttcaagtccagtccccttctataaaaatgagccttgagtcctacgggaagagctatggtagcatggtccccagcaatgagaactgtcgcaagacttatgataccactgtggatgacaatgagagctattacaagagttacaccagcacccagaccagcagcaagagctttctcaagagctatgacagcagcaccagtgccagtgaggcctatgggaagagttactgcactaccagcaacagcagcattacctataagaagagttacggcagcaccagtagctctgacacctgccagaagagttttgtcagcagctgcactgacgaggaacctgctgagcctgaagacatggagcgctttgaggaaatggttgtgaaagtgctgatcaagctgcaggcggtgcaggccatgtaccagataagccaggaggaacacagccagctgcaagagcagatggaaaagttactggccaagcagaaagacctgaaggaagagctggatgcctgtgaaagggagttcaaggagtgcatggaatgccttgaaaagcccatggccccccagaacgacaagaatgagatcaaagaactgcagaccaagctgcgggagctgcagctgcaataccaggctagcatggatgagcaggggcagcttctggtagtgcaggagcagctggaggggcagctgcagtgctgccaggaggagctccgccagctcagggagaagaggccttctgttgtcaaagaagcccgggggaagaatgctaataagaacatgaacaagaatgccaatggggttaaaatgaaaaaggtgaccaagccatgctcggatacttctgagagcgaccttgagaccagaaagagtctggaggtagtgctgtactacaaggccagccagaggaaattagatggactagcaaaagaggaggaaaagaaagaggagatggaggaggaaaaaaaggaagtgaaagaggaagcaaaggagcagtgtggggatgagctagttgctgagccagcagatcctgaggaagctaaatccacagaagatcaggaggaaaatgaagaggacaaagaggaagaggagaaggaagaagacagtgaagaggaggaagatgacgccgactcttcccttgaaagtcccgaagaaaataaccccctcagactttccgagagcaaaaagaacatgtttgggttgtggaagcctatggtattcttggctattgcagctgtggctctgtatgtgttacccaacatgcgacagcaggagtcagagttctgcctcatggagtgatggcagaccttggccagcgcgagggcagatccccagtggccaccaccctcagctttgggcaggacacactgtgccagaaccctccccatatgttccatgtgtccccatctcctcagcctcagtcacccaggctgaaaaggcttgtggggagcggctgacttccatctcctgccttgtgtaagaacctgagttccttgtaattaaatatcaactgaattaaaaaaaaaaaaaaaaaa

SEQ ID NO: 6 - protein, 1154 aa, Homo sapiens
MEAGAGAGAGAAGWSCPGPGPTVTTLGSYEASEGCERKKGQRWGSLERRGMQAMEGEVLLPALYEEEEEEEEEEEEVEEEEEQVQKGGSVGSLSVNKHRGLSLTETELEELRAQVLQLVAELEETRELAGQHEDDSLELQGLLEDERLASAQQAEVFTKQIQQLQGELRSLREEISLLEHEKESELKEIEQELHLAQAEIQSLRQAAEDSATEHESDIASLQEDLCRMQNELEDMERIRGDYEMEIASLRAEMEMKSSEPSGSLGLSDYSGLQEELQELRERYHFLNEEYRALQESNSSLTGQLADLESERTQRATERWLQSQTLSMTSAESQTSEMDFLEPDPEMQLLRQQLRDAEEQMHGMKNKCQELCCELEELQHHRQVSEEEQRRLQRELKCAQNEVLRFQTSHSVTQELLCRLQKLHLQHQNVTCEKEKLLERQQQLQEELQCHEAELQHLRDTVASFKESNEKDTETHAQLQEMKQLYQASKDELERQKHMYDQLEQDLLLCQLELKELKASHPIPEDKGKCANKCDTLLSRLTELQEKYKASQKEMGQLQMEQCELLEDQRRMQEEQGQLQEELHRLTLPLPKSGLLLKSQELLTKLEDLCELQLLYQGMQEEQKKLIQNQDCVLKEQLEIHEELRRFKESHFQEVLENPDDSKLAKSSKCNRNKQSKLLMEQMQALQVMYDAGQAKQELLQQEQGRLLEERKRLQADLQLCLEEMQLLQVQSPSIKMSLESYGKSYGSMVPSNENCRKTYDTTVDDNESYYKSYTSTQTSSKSFLKSYDSSTSASEAYGKSYCTTSNSSITYKKSYGSTSSSDTCQKSFVSSCTDEEPAEPEDMERFEEMVVKVLIKLQAVQAMYQISQEEHSQLQEQMEKLLAKQKDLKEELDACEREFKECMECLEKPMAPQNDKNEIKELQTKLRELQLQYQASMDEQGQLLVVQEQLEGQLQCCQEELRQLREKRPSVVKEARGKNANKNMNKNANGVKMKKVTKPCSDTSESDLETRKSLEVVLYYKASQRKLDGLAKEEEKKEEMEEEKKEVKEEAKEQCGDELVAEPADPEEAKSTEDQEENEEDKEEEEKEEDSEEEEDDADSSLESPEENNPLRLSESKKNMFGLWKPMVFLAIAAVALYVLPNMRQQESEFCLME

SEQ ID NO: 7 - DNA, 3692 bp, Homo sapiens
  CDS (14)..(3637)
ggcacgagcggtcatggaggcgggcgccggagccggcgcgggagccgcgggctggagctgcccgggcccaggacccacagtgaccactctaggctcctatgaggcttccgagggctgtgagaggaagaagggccaacgctgggggtccctggaacgacgggggatgcaagctatggagggggaggtgttactcccagctctctatgaggaggaagaggaagaggaagaggaggaagaagaggtggaagaagaagaagaacaagtgcagaaaggtggcagtgttggctctctgtcagtcaacaagcaccggggactgagcctcacggagacagagctggaggagctgcgggctcaggtgctgcagctggtggcagaactggaggagacccgggaactggcagggcagcatgaggatgactccttggagctacaggggctcctggaggatgaacggctagccagcgcccagcaggcagaggtgttcaccaagcagatccagcagctccaaggtgagctgcgttctctacgggaggagatttccctgttagagcatgagaaagaaagcgaacttaaggaaatagaacaggaattgcatttggcccaggctgagatccagagtctgcggcaagcagcagaggattccgcaactgaacatgagagtgacatagcatccctgcaggaggatctctgccggatgcagaatgaacttgaagacatggaacgcattcggggagattatgagatggagatcgcctccctccgtgcagaaatggaaatgaagagctctgaaccatccggtagtttaggtctctcagattactctgggttacaagaagaactgcaggagctgcgggaacgctaccatttcctgaatgaggaataccgggccctgcaggagagcaacagcagcctcacggggcagcttgcagatctggagagtgagaggacacagagagcaacagagagatggctgcagtcccaaacactgagtatgacgtcagcagagtctcagacttcagaaatggatttcttagagcctgatcctgaaatgcagttgttacggcagcagctacgggatgctgaagagcagatgcatggcatgaagaacaagtgtcaggaattgtgttgtgagttggaagagctacagcatcatcgccaggtcagtgaggaggagcagaggcggctgcagagggagctcaagtgtgctcagaatgaggtgcttcggtttcagacctcccacagtgtcacccagaatgaggagctgaagtccagactctgtaccctgcagaaaaaatatgatactagccaggatgagcagaacgagctcttgaagatgcagctgcaacttcagactgagctccggcagctcaaagtcatgaaatccacacttgtagaaaaccagagtgagaaggagttactgtgccggctgcagaagctgcacctccagcaccagaacgtcacatgtgagaaggaaaagctgctggaacggcagcagcagctgcaggaggagctgcagtgccatgaggcagagctgcagcacctcagggatacggtggcctccttcaaagagagcaatgagaaggacacagagacgcacgctcagcttcaggagatgaagcagctgtaccaggccagcaaggacgagctggagcggcagaagcacatgtatgaccagctggagcaggacctcctgctctgccagctggagctgaaagagctcaaggcctcccaccccattccggaggacaaaggaaagtgtgctaataagtgtgacacactgctgtccagactgacagaattgcaggaaaagtacaaggccagccagaaggagatggggcagctgcagatggagcagtgtgagctcctggaggatcagaggaggatgcaggaggagcagggccagctgcaggaagagctgcacaggctcacactgccactgccaaagagtggcctcttactcaagagtcaggagctactcaccaagttagaagacctgtgtgagctgcagctgctctaccaaggcatgcaggaggaacagaagaagctgatacagaaccaagactgtgtattaaaagaacaattagagatccacgaagagctgcgacgtttcaaagagtctcatttccaggaagtgttggagaatcccgatgattccaaattggctaagtcctccaaatgtaatcgaaacaagcaatccaagctgctcatggagcagatgcaggccctgcaggtgatgtatgacgccggtcaggcgaagcaggagctcttgcagcaagagcaagggaggctcctagaggagcggaagaggctgcaggcagacttgcagctctgcctggaagaaatgcagctgcttcaagtccagtccccttctataaaaatgagccttgagtcctacgggaagagctatggtagcatggtccccagcaatgagaactgtcgcaagacttatgataccactgtggatgacaatgagagctattacaagagttacaccagcacccagaccagcagcaagagctttctcaagagctatgacagcagcaccagtgccagtgaggcctatgggaagagttactgcactaccagcaacagcagcattacctataagaagagttacggcagcaccagtagctctgacacctgccagaagagttttgtcagcagctgcactgacgaggaacctgctgagcctgaagacatggagcgctttgaggaaatggttgtgaaagtgctgatcaagctgcaggcggtgcaggccatgtaccagataagccaggaggaacacagccagctgcaagagcagatggaaaagttactggccaagcagaaagacctgaaggaagagctggatgcctgtgaaagggagttcaaggagtgcatggaatgccttgaaaagcccatggccccccagaacgacaagaatgagatcaaagaactgcagaccaagctgcgggagctgcagctgcaataccaggctagcatggatgagcaggggcagcttctggtagtgcaggagcagctggaggggcagctgcagtgctgccaggaggagctccgccagctcagggagaagaggccttctgttgtcaaagaagcccgggggaagaatgctaataagaacatgaacaagaatgccaatggggttaaaatgaaaaaggtgaccaagccatgctcggatacttctgagagcgaccttgagaccagaaagaagatcaggaggaaaatgaagaggacaaagaggaagaggagaaggaagaagacagtgaagaggaggaagatgacgccgactcttcccttgaaagtcccgaagaaaataaccccctcagactttccgagagcaaaaagaacatgtttgggttgtggaagcctatggtattcttggctattgcagctgtggctctgtatgtgttacccaacatgcgacagcaggagtcagagttctgcctcatggagtgatggcagaccttggccagcgcgagggcagatccccagtggccaccaccctcagctttgggcaggacacactgtgccagaaccctccccatatgttccatgtgtccccatctcctcagcctcagtcacccaggctgaaaaggcttgtggggagcggctgacttccatctcctgccttgtgtaagaacctgagttccttgtaattaaatatcaactgaattaaaaaaaaaaaaaaaaaa

SEQ ID NO: 8 - protein, 1207 aa, Homo sapiens
MEAGAGAGAGAAGWSCPGPGPTVTTLGSYEASEGCERKKGQRWGSLERRGMQAMEGEVLLPALYEEEEEEEEEEEEVEEEEEQVQKGGSVGSLSVNKHRGLSLTETELEELRAQVLQLVAELEETRELAGQHEDDSLELQGLLEDERLASAQQAEVFTKQIQQLQGELRSLREEISLLEHEKESELKEIEQELHLAQAEIQSLRQAAEDSATEHESDIASLQEDLCRMQNELEDMERIRGDYEMEIASLRAEMEMKSSEPSGSLGLSDYSGLQEELQELRERYHFLNEEYRALQESNSSLTGQLADLESERTQRATERWLQSQTLSMTSAESQTSEMDFLEPDPEMQLLRQQLRDAEEQMHGMKNKCQELCCELEELQHHRQVSEEEQRRLQRELKCAQNEVLRFQTSHSVTQNEELKSRLCTLQKKYDTSQDEQNELLKMQLQLQTELRQLKVMKSTLVENQSEKELLCRLQKLHLQHQNVTCEKEKLLERQQQLQEELQCHEAELQHLRDTVASFKESNEKDTETHAQLQEMKQLYQASKDELERQKHMYDQLEQDLLLCQLELKELKASHPIPEDKGKCANKCDTLLSRLTELQEKYKASQKEMGQLQMEQCELLEDQRRMQEEQGQLQEELHRLTLPLPKSGLLLKSQELLTKLEDLCELQLLYQGMQEEQKKLIQNQDCVLKEQLEIHEELRRFKESHFQEVLENPDDSKLAKSSKCNRNKQSKLLMEQMQALQVMYDAGQAKQELLQQEQGRLLEERKRLQADLQLCLEEMQLLQVQSPSIKMSLESYGKSYGSMVPSNENCRKTYDTTVDDNESYYKSYTSTQTSSKSFLKSYDSSTSASEAYGKSYCTTSNSSITYKKSYGSTSSSDTCQKSFVSSCTDEEPAEPEDMERFEEMVVKVLIKLQAVQAMYQISQEEHSQLQEQMEKLLAKQKDLKEELDACEREFKECMECLEKPMAPQNDKNEIKELQTKLRELQLQYQASMDEQGQLLVVQEQLEGQLQCCQEELRQLREKRPSVVKEARGKNANKNMNKNANGVKMKKVTKPCSDTSESDLETRKKIRRKMKRTKRKRRRKKTVKRRKMTPTLPLKVPKKITPSDFPRAKRTCLGCGSLWYSWLLQLWLCMCYPTCDSRSQSSASWSDGRPWPARGQIPSGHHPQLWAGHTVPEPSPYVPCVPISSASVTQAEKACGERLTSISCLV

SEQ ID NO: 9 - DNA, 3873 bp, Homo sapiens
  CDS (14)..(3637)
ggcacgagcggtcatggaggcgggcgccggagccggcgcgggagccgcgggctggagctgcccgggcccaggacccacagtgaccactctaggctcctatgaggcttccgagggctgtgagaggaagaagggccaacgctgggggtccctggaacgacgggggatgcaagctatggagggggaggtgttactcccagctctctatgaggaggaagaggaagaggaagaggaggaagaagaggtggaagaagaagaagaacaagtgcagaaaggtggcagtgttggctctctgtcagtcaacaagcaccggggactgagcctcacggagacagagctggaggagctgcgggctcaggtgctgcagctggtggcagaactggaggagacccgggaactggcagggcagcatgaggatgactccttggagctacaggggctcctggaggatgaacggctagccagcgcccagcaggcagaggtgttcaccaagcagatccagcagctccaaggtgagctgcgttctctacgggaggagatttccctgttagagcatgagaaagaaagcgaacttaaggaaatagaacaggaattgcatttggcccaggctgagatccagagtctgcggcaagcagcagaggattccgcaactgaacatgagagtgacatagcatccctgcaggaggatctctgccggatgcagaatgaacttgaagacatggaacgcattcggggagattatgagatggagatcgcctccctccgtgcagaaatggaaatgaagagctctgaaccatccggtagtttaggtctctcagattactctgggttacaagaagaactgcaggagctgcgggaacgctaccatttcctgaatgaggaataccgggccctgcaggagagcaacagcagcctcacggggcagcttgcagatctggagagtgagaggacacagagagcaacagagagatggctgcagtcccaaacactgagtatgacgtcagcagagtctcagacttcagaaatggatttcttagagcctgatcctgaaatgcagttgttacggcagcagctacgggatgctgaagagcagatgcatggcatgaagaacaagtgtcaggaattgtgttgtgagttggaagagctacagcatcatcgccaggtcagtgaggaggagcagaggcggctgcagagggagctcaagtgtgctcagaatgaggtgcttcggtttcagacctcccacagtgtcacccagaatgaggagctgaagtccagactctgtaccctgcagaaaaaatatgatactagccaggatgagcagaacgagctcttgaagatgcagctgcaacttcagactgagctccggcagctcaaagtcatgaaatccacacttgtagaaaaccagagtgagaaggagttactgtgccggctgcagaagctgcacctccagcaccagaacgtcacatgtgagaaggaaaagctgctggaacggcagcagcagctgcaggaggagctgcagtgccatgaggcagagctgcagcacctcagggatacggtggcctccttcaaagagagcaatgagaaggacacagagacgcacgctcagcttcaggagatgaagcagctgtaccaggccagcaaggacgagctggagcggcagaagcacatgtatgaccagctggagcaggacctcctgctctgccagctggagctgaaagagctcaaggcctcccaccccattccggaggacaaaggaaagtgtgctaataagtgtgacacactgctgtccagactgacagaattgcaggaaaagtacaaggccagccagaaggagatggggcagctgcagatggagcagtgtgagctcctggaggatcagaggaggatgcaggaggagcagggccagctgcaggaagagctgcacaggctcacactgccactgccaaagagtggcctcttactcaagagtcaggagctactcaccaagttagaagacctgtgtgagctgcagctgctctaccaaggcatgcaggaggaacagaagaagctgatacagaaccaagactgtgtattaaaagaacaattagagatccacgaagagctgcgacgtttcaaagagtctcatttccaggaagtgttggagaatcccgatgattccaaattggctaagtcctccaaatgtaatcgaaacaagcaatccaagctgctcatggagcagatgcaggccctgcaggtgatgtatgacgccggtcaggcgaagcaggagctcttgcagcaagagcaagggaggctcctagaggagcggaagaggctgcaggcagacttgcagctctgcctggaagaaatgcagctgcttcaagtccagtccccttctataaaaatgagccttgagtcctacgggaagagctatggtagcatggtccccagcaatgagaactgtcgcaagacttatgataccactgtggatgacaatgagagctattacaagagttacaccagcacccagaccagcagcaagagctttctcaagagctatgacagcagcaccagtgccagtgaggcctatgggaagagttactgcactaccagcaacagcagcattacctataagaagagttacggcagcaccagtagctctgacacctgccagaagagttttgtcagcagctgcactgacgaggaacctgctgagcctgaagacatggagcgctttgaggaaatggttgtgaaagtgctgatcaagctgcaggcggtgcaggccatgtaccagataagccaggaggaacacagccagctgcaagagcagatggaaaagttactggccaagcagaaagacctgaaggaagagctggatgcctgtgaaagggagttcaaggagtgcatggaatgccttgaaaagcccatggccccccagaacgacaagaatgagatcaaagaactgcagaccaagctgcgggagctgcagctgcaataccaggctagcatggatgagcaggggcagcttctggtagtgcaggagcagctggaggggcagctgcagtgctgccaggaggagctccgccagctcagggagaagaggccttctgttgtcaaagaagcccgggggaagaatgctaataagaacatgaacaagaatgccaatggggttaaaatgaaaaaggtgaccaagccatgctcggatacttctgagagcgaccttgagaccagaaagagtctggaggtagtgctgtactacaaggccagccagaggaaattagatggactagcaaaagaggaggaaaagaaagaggagatggaggaggaaaaaaaggaagtgaaagaggaagcaaaggagcagtgtggggatgagctagttgctgagccagcagatcctgaggaagctaaatccacagaagatcaggaggaaaatgaagaggacaaagaggaagaggagaaggaagaagacagtgaagaggaggaagatgacgccgactcttcccttgaaagtcccgaagaaaataaccccctcagactttccgagagcaaaaagaacatgtttgggttgtggaagcctatggtattcttggctattgcagctgtggctctgtatgtgttacccaacatgcgacagcaggagtcagagttctgcctcatggagtgatggcagaccttggccagcgcgagggcagatccccagtggccaccaccctcagctttgggcaggacacactgtgccagaaccctccccatatgttccatgtgtccccatctcctcagcctcagtcacccaggctgaaaaggcttgtggggagcggctgacttccatctcctgccttgtgtaagaacctgagttccttgtaattaaatatcaactgaattaaaaaaaaaaaaaaaaaa

SEQ ID NO: 10 - protein, 1207 aa, Homo sapiens
MEAGAGAGAGAAGWSCPGPGPTVTTLGSYEASEGCERKKGQRWGSLERRGMQAMEGEVLLPALYEEEEEEEEEEEEVEEEEEQVQKGGSVGSLSVNKHRGLSLTETELEELRAQVLQLVAELEETRELAGQHEDDSLELQGLLEDERLASAQQAEVFTKQIQQLQGELRSLREEISLLEHEKESELKEIEQELHLAQAEIQSLRQAAEDSATEHESDIASLQEDLCRMQNELEDMERIRGDYEMEIASLRAEMEMKSSEPSGSLGLSDYSGLQEELQELRERYHFLNEEYRALQESNSSLTGQLADLESERTQRATERWLQSQTLSMTSAESQTSEMDFLEPDPEMQLLRQQLRDAEEQMHGMKNKCQELCCELEELQHHRQVSEEEQRRLQRELKCAQNEVLRFQTSHSVTQNEELKSRLCTLQKKYDTSQDEQNELLKMQLQLQTELRQLKVMKSTLVENQSEKELLCRLQKLHLQHQNVTCEKEKLLERQQQLQEELQCHEAELQHLRDTVASFKESNEKDTETHAQLQEMKQLYQASKDELERQKHMYDQLEQDLLLCQLELKELKASHPIPEDKGKCANKCDTLLSRLTELQEKYKASQKEMGQLQMEQCELLEDQRRMQEEQGQLQEELHRLTLPLPKSGLLLKSQELLTKLEDLCELQLLYQGMQEEQKKLIQNQDCVLKEQLEIHEELRRFKESHFQEVLENPDDSKLAKSSKCNRNKQSKLLMEQMQALQVMYDAGQAKQELLQQEQGRLLEERKRLQADLQLCLEEMQLLQVQSPSIKMSLESYGKSYGSMVPSNENCRKTYDTTVDDNESYYKSYTSTQTSSKSFLKSYDSSTSASEAYGKSYCTTSNSSITYKKSYGSTSSSDTCQKSFVSSCTDEEPAEPEDMERFEEMVVKVLIKLQAVQAMYQISQEEHSQLQEQMEKLLAKQKDLKEELDACEREFKECMECLEKPMAPQNDKNEIKELQTKLRELQLQYQASMDEQGQLLVVQEQLEGQLQCCQEELRQLREKRPSVVKEARGKNANKNMNKNANGVKMKKVTKPCSDTSESDLETRKSLEVVLYYKASQRKLDGLAKEEEKKEEMEEEKKEVKEEAKEQCGDELVAEPADPEEAKSTEDQEENEEDKEEEEKEEDSEEEEDDADSSLESPEENNPLRLSESKKNMFGLWKPMVFLAIAAVALYVLPNMRQQESEFCLME

SEQ ID NO: 11 - DNA, 3847 bp, Homo sapiens
  CDS (211)..(2883)
gcggaggtggagtacaacgggggcttccacctggccatcgacgtggacctggtcttcggcaagtccgcctacttgtttgtcaagctgtcccgcgtggtggggaggctgcgcttggtctttacgcgcgtgcccttcacccactggttcttctccttcgtggaagacccgctgatcgacttcgaggtgcgctcgcagtttgaagggcggcccatgccccagctcacctccatcatcgtcaaccagctcaagaagatcatcaagcgcaagcacaccctaccgaattacaagatcaggtttaagccgttttttccataccagaccttgcaaggatttgaagaagatgaagagcatatccatatacaacaatgggcacttactgaaggccgtcttaaagttacgttgttagaatgtagcaggttactcatttttggatcctatgacagagaggcaaatgttcattgcacacttgagttaagcagtagtgtttgggaagaaaaacagaggagttctattaagacggttgaattaataaaaggaaatttacaaagtgttggacttacacttcgtcttgtccagtcaactgatgggtatgctgggcacgtcatcattgaaactgtggctccaaactcgcctgctgcaattgcagatcttcagcggggagatcgacttatcgccattggaggtgtgaaaatcacatcaacactgcaagtgttgaagcttatcaagcaggctggtgaccgagtcctggtgtactatgaaaggcctgttggccagagtaatcaaggtgcagtgctgcaagataactttggccagttggaagaaaactttttgtcaagctcatgccaatcgggttatgaagaggaagctgccgggttgacagtagatactgaaagtagagagctggattctgaatttgaagacttggcaagtgatgtcagagcacaaaatgagttcaaagatgaggcacaatcattaagtcatagtcccaaacgtgttccaacaacactttctattaaaccccttggagctatatcaccagttttaaaccgtaaattagctgtaggaagtcacccactaccaccgaaaattcagtccaaagatggaaataaacctccacccctaaaaacttctgagataacagacccagcacaagtgtcaaaaccaacccaaggatctgctttcaaaccacctgtgccaccacgaccacaagcgaaagttcctttgccttccgccgatgctccaaatcaggcagaaccagatgttctcgttgaaaagccagagaaggtggtgccacctcctcttgtagataaatctgctgaaaagcaagcaaaaaatgtggatgccatagacgatgcagctgcacctaagcaatttttagcaaagcaagaagtggccaaagatgtcacttcagaaactttctgccctactaaggacagttcggacgaccgtcaaacatgggaatcatcagaaattctttatcgtaataagctaggaaaatggacaagaaccagagcatcctgtttgtttgacatagaagcctgtcacaggtacttaaacattgcattgtggtgcagggatcctttcaagttgggaggtctcatctgtttggggcatgttagtttaaaacttgaagatgtggctttaggatgcctagctacatcaaacacggaatacctttccaaattgagactggaagccccctcacctaaggctatagtcactagaaccgcactacgcaatctgagtatgcaaaagggattcaatgacaaattttgctatggtgacattactattcacttcaaatatttgaaagaaggagaatcagaccaccatgtagttactaacgtagaaaaagaaaaagaaccccatttggttgaagaagtttctgttctccctaaagaggagcaatttgttggacagatgggtttaacagaaaacaaacacagttttcaggatactcagttccagaacccaacatggtgtgactactgtaagaaaaaagtttggactaaagcagcttcccagtgtatgttttgtgcttatgtttgccataaaaaatgtcaagaaaagtgtctagctgagacttctgtttgtggagcaactgataggcgaatagacaggacactgaaaaaccttaggctggaaggacaggaaaccctcttaggcctgcctcctcgtgttgatgctgaagctagcaagtcagtcaataaaacaacaggtttgacaaggcatattatcaatactagttctcgtttattaaatttgcgtcaagtctctaaaactcgcctttctgaaccaggaaccgatctcgtagaaccttcaccaaaacacacacccaacacgtcagacaacgaaggcagtgacacggaggtctgtggtccaaacagtccttctaaacggggaaacagcacaggaataaagttagtgagaaaagagggtggtctggatgacagtgttttcattgcagttaaagaaattggtcgtgatctgtacaggggcttgcctacagaggaaaggatccagaaactagagttcatgttggataagctacagaatgaaattgatcaggagttggaacacaataattcccttgttagagaagaaaaagagacaactgatacaaggaaaaaatcacttctttctgctgccttagctaaatcaggtgaaaggctacaagctctaacacttcttatgattcactacagagcaggcattgaagatatagaaactttagaaagtctgtctttagaccagcactccaaaaaaataagcaagtacacagatgatacagaagaagaccttgataatgaaataagccaactaatagactctcagccattcagcagcatatcagatgacttatttggcccatccgagtctgtgtagcagacaggtctatttaaactttcaaatgaacagggtaaagttgcatctaaagtaccacagatacaaccatgtttaaatcctcgtatgcactctggcctgcttctccagttacttgcttgtgtaagaacaaaaatgagaaaggttgttttccagtaaaaacatgaccagcttactaattggttgttttggattgcatttatagctatgcttttttgggtttatactgggaatttatttttactaaattatttaacttttctaattatgtaattatgtaagctagcttttcatgtttatgtatgtatggtgtccccttgtgttatttttcttcctcttggtttttgaattagtgttaaatagaatactgtctagattcttaaaatattttcatttccatcatggttataacaaatttgctgcatgcccaaactgacaacagcantcactgagggaacaggttttgaatctttcttttgtgttatgaagtttatcgtctctacttgcttgagatttttgttattttgggggtttgggggtgctttttgttttgtttttgccaaatgtaacatgaaagcagatgctgcagctttagtctgttatgctgatttagtaaaaaaaaattttttacatatattgcttgctttcgatgcttctgtgaaatttttttctaaagcttttgtgcagctgtatggtaaaaatatggtgattaatttgaagagcttacattgaaagacaatgtaataggaaataaatgtagattgcagttggtcaagaattttgtagagaggataacaagacttaattactgaaaaacagtaacatagcattttgaaatataatcttttaaaatattgatgctttccttttaaatggaaatttaaattttataattaaaagtttaaacatttatgataattttcctcatcagttctcccataggaaataaagcatgtgaaagggt

SEQ ID NO: 12 - protein, 891 aa, Homo sapiens
MPQLTSIIVNQLKKIIKRKHTLPNYKIRFKPFFPYQTLQGFEEDEEHIHIQQWALTEGRLKVTLLECSRLLIFGSYDREANVHCTLELSSSVWEEKQRSSIKTVELIKGNLQSVGLTLRLVQSTDGYAGHVIIETVAPNSPAAIADLQRGDRLIAIGGVKITSTLQVLKLIKQAGDRVLVYYERPVGQSNQGAVLQDNFGQLEENFLSSSCQSGYEEEAAGLTVDTESRELDSEFEDLASDVRAQNEFKDEAQSLSHSPKRVPTTLSIKPLGAISPVLNRKLAVGSHPLPPKIQSKDGNKPPPLKTSEITDPAQVSKPTQGSAFKPPVPPRPQAKVPLPSADAPNQAEPDVLVEKPEKVVPPPLVDKSAEKQAKNVDAIDDAAAPKQFLAKQEVAKDVTSETFCPTKDSSDDRQTWESSEILYRNKLGKWTRTRASCLFDIEACHRYLNIALWCRDPFKLGGLICLGHVSLKLEDVALGCLATSNTEYLSKLRLEAPSPKAIVTRTALRNLSMQKGFNDKFCYGDITIHFKYLKEGESDHHVVTNVEKEKEPHLVEEVSVLPKEEQFVGQMGLTENKHSFQDTQFQNPTWCDYCKKKVWTKAASQCMFCAYVCHKKCQEKCLAETSVCGATDRRIDRTLKNLRLEGQETLLGLPPRVDAEASKSVNKTTGLTRHIINTSSRLLNLRQVSKTRLSEPGTDLVEPSPKHTPNTSDNEGSDTEVCGPNSPSKRGNSTGIKLVRKEGGLDDSVFIAVKEIGRDLYRGLPTEERIQKLEFMLDKLQNEIDQELEHNNSLVREEKETTDTRKKSLLSAALAKSGERLQALTLLMIHYRAGIEDIETLESLSLDQHSKKISKYTDDTEEDLDNEISQLIDSQPFSSISDDLFGPSESV

SEQ ID NO: 13 - DNA, 20 bp, Artificial Sequence
  Description of Artificial SequenceT3
aattaaccctcactaaaggg

SEQ ID NO: 14 - DNA, 22 bp, Artificial Sequence
  Description of Artificial SequenceT7
gtaatacgactcactatagggc

SEQ ID NO: 15 - DNA, 22 bp, Artificial Sequence
  Description of Artificial SequenceKU-GB-2 Sense Primer
agagcgaccttgagaccagaaa

SEQ ID NO: 16 - DNA, 22 bp, Artificial Sequence
  Description of Artificial SequenceKU-GB-2 Antisense Primer
acacaaggcaggagatggaagt

SEQ ID NO: 17 - DNA, 20 bp, Artificial Sequence
  Description of Artificial SequenceKU-GB-5 Sense Primer
aaaactcgcctttctgaacc

SEQ ID NO: 18 - DNA, 20 bp, Artificial Sequence
  Description of Artificial SequenceKU-GB-5 Antisense Primer
agcaagtaactggagaagca

SEQ ID NO: 19 - DNA, 25 bp, Artificial Sequence
  Description of Artificial SequenceBeta-actin Sense Primer
gtcgacaacggctccggcatgtgca

SEQ ID NO: 20 - DNA, 25 bp, Artificial Sequence
  Description of Artificial SequenceBeta-actin Antisense Primer
ggatcttcatgaggtagtcagtcag

(19) United States
(12) Patent Application Publication
(10) Pub. No.: US 2004/0072265 A1
(43) Pub. Date: Apr. 15, 2004

(54) Human glioma antigen and process for preparing the same

(76) Inventors: Masahiro Toda, Yokohama-shi (JP); Yutaka Kawakami, Yokahama-shi (JP); Takeshi Kawase, 1-chome Ichikawa-shi (JP); Yukihiko Iizuka, Suginami-ku (JP)

Correspondence Address: MORGAN & FINNEGAN, L.L.P., 345 PARK AVENUE, NEW YORK, NY, 10154, US

(21) Appl. No.: 10/250,823

(22) PCT Filed: Nov. 30, 2001
(86) PCT No.: PCT/JP01/10505

(30) Foreign Application Priority Data
Jan. 9, 2001 (JP) 2001-1965

(51) Int. Cl. G01N 33/574; C12P 21/02; C12N 1/21

(52) U.S. Cl. 435/7.23; 435/69.3; 435/252.33; 435/320.1

(57) ABSTRACT

The present invention provides a human glioma antigen or a method for preparing the same, which can be applied to diagnosis and therapy of glioma. A glioma antigen or a glioma antigen gene is prepared by a process comprising the steps of: total RNA is extracted from a glioma cell line and cDNA was synthesized; λ phage cDNA library, which was constructed by infection of E. coli caused by introducing said cDNA into λ phage vector, and the sera of glioma patients are made to react; positive clones to which antibodies in the serum reacted are detected with the use of labeled anti-IgG antibodies; screening is repeated several times for the detected positive clones; antigens are isolated from positive clones confirmed to be antibody-reactive; serum screening is conducted by using the isolated antigens, the sera of glioma patients and healthy persons.

### TECHNICAL FIELD

The present invention relates to: a glioma antigen or its gene expressing and showing immunoreaction in a human malignant brain tumor, i.e. glioma; a method for preparing said glioma antigen or its gene; a diagnostic drug for detecting a glioma; a detecting and diagnostic probe; an antibody against a glioma antigen and its gene; a screening method for a promoter or a suppressor for the immunity-inducing activity using a glioma antigen; etc.

### BACKGROUND ART

There has long been expectations on immunotherapies and many attempts have been taken. However, sufficient anti-tumor effects have not been materialized. Cancer immunotherapies have historically been handled with a central focus on the non-specific immunotherapy, however, it has become evident in recent years that a T cell plays an important role at tumor rejection in vivo in human melanoma. Moreover, many efforts have been drawn to the isolation of a T cell recognition tumor antigen which would possibly induce cytotoxic T lymphocyte (CTL) and determining of a MHC Class I-restricted epitope, since when MAGE-1 antigen (Science 254, 1643-7, 1991) was reported by a Belgian group in 1991. The present inventors isolated a CD8+T cell recognition by the cDNA expression cloning method by using a tumor reactive T cell of melanoma (Proc. Natl. Acad. Sci. USA 91, 3515-3519, 1994, Proc. Natl. Acad. Sci. USA 91, 6458-6462, 1994, J. Exp. Med. 180, 347-352, 1994, J. Immunol. 154, 3961-3968, 1995, Immunologic Res. 16, 313-340, 1997), and they reported that an anti-tumor effect was recognized for part of melanoma by this specific immunothoerapy using said antigen (Microbiology Immunology 42, 803-813, 1998, Kawakami, Y., P. F. Robbins, R F. Wang. et al. Identification of Melanoma antigens by T lymphocytes and their use in the immunotherapy of cancer. In Principle and Practice of Oncology. Update. V. DeVita, S. Hellman, S. A. Rosenberg eds. J.B. Lippincott Co. Philadelphia, p1-20, 1996, Nature Med. 4, 321, 1998). It is further found that the antigen recognized by T cell is a peptide bound to a MHC molecule, which is not necessarily derived from a cell surface protein, and intranuclear or cytoplasm protein can be recognized as an antigen (Immunity 10, 281-7, 1999).

In 1995, groups of Pfreundschuh in Germany and Old et al. in U.S. reported the SEREX method (serological identification of recombinant cDNA expression cloning; Proc. Natl. Acad. Sci. USA 92, 11810-11813, 1995) for detecting cancer antigen protein which is recognized by an IgG antibody in sera of cancer patients. The method is applicable for cancer wherein the establishment of cell line is difficult, and does not necessarily require a tumor and a CTL. Many tumor antigens have been isolated by applying this method, and it has been reported that this method is also effective for detecting antigens recognized by celluler immunity since antigens such as MAGE-1 and tyrosinase which induce CTL were found to be included in the antigens isolated by this method. It is further reported that the cancer antigens recognized by an IgG antibody of a patient was isolated in melanoma, renal cancer, esophageal cancer, colon cancer, lung cancer, etc., by applying the aforementioned method (Int. J. Cancer 72, 965-971, 1997, Cancer Res. 58, 1034-1041, 1998, Int. J. Cancer 29, 652-658, 1998, Int. J. Oncol. 14, 703-708, 1999, Cancer Res. 56, 4766-4772, 1996, Hum. Mol. Genet 6,33-39, 1997). These antigens include important antigens such as a CT (cancer-testis) antigen (SSX2/HOM-MEL-40, NY-ESO-1, SCP-1, CT7 etc.), a differentiated antigen (galectin-4/NY-CO-27), a mutant antigen (p53 etc.). Among these antigens, HLA-A2-restricted epitope is particularly determined with NY-ESO-1. Further, the antigens isolated by the SEREX method include molecules such as e1F-4γ or HER2/neu which over-express in cancer, and which possibly relate to malignant alteration. There is a possibility that these molecules over-expressing in cancer can be applied not only to a therapy but also to a marker for diagnosis or for possibly projecting recurrence and prognosis.

On the other hand, central nervous system has been known as a venue for immunological tolerance. However, it has recently been appeared that an activated lymphocyte reacts with an intracerebral antigen after transiting through a blood brain barrier and invading into brain (J. Immunol. 158, 2318-26, 1997, J. Neurosci. 18,5804-16,1998). The present inventors further indicated the possibility that a specific immunity-inducing against a tumor antigen in a brain could be a new effective therapy method (Neuro-Oncology 1, S105, 1999). Image analyses by MRI et al. and histopathological analyses by using a specimen obtained through an operation are presently focused on for the diagnostic methods for detecting glioma, malignant brain tumor. However, as the genes related to the generation of glioma are becoming evident due to the progress of molecular cell biology in recent years, the possibility of genetic diagnosis wherein said genes are analyzed is also indicated (Glia. 15, 308-27, 1995).

At present it is still the case for cancer, which is currently the primary cause of death, that most of advanced cancers cannot be remedied in spite of the improvement in their mechanisms of generation, diagnostic methods, and therapy methods. In particular, malignant brain tumors are extremely difficult to be remedied, and there has been no effective therapy to date in spite of the improvement in brain surgery operations and radiotherapies in recent years. It is required to develop a new and early diagnostic method and a therapy method in order to improve such situation. The object of the present invention is to provide a human glioma antigen which can be applied to diagnosis and therapy of glioma, a gene encoding such antigen, and a method for preparing the same.

The present inventors have made a keen study to attain the objects mentioned above, and the present inventors constructed cDNA by using mRNA obtained from a glioma cell line, and this cDNA was introduced into λ phage vector to infect Escherichia coli, and by using thus constructed cDNA library (6.0×10⁵ λ phage cDNA clones) derived from a glioma cell line, the present inventors screened the serum of each glioma patient, and found that a glioma antigen which reacts only to the sera of glioma patients and does not react to the sera of healthy persons, and which selectively expresses in cancer cells including a glioma cell line is present, and that an immune system recognizes the protein wherein glioma expresses. Here, the present invention is completed.

### DISCLOSURE OF THE INVENTION

The present invention relates to: a method for preparing a glioma antigen and/or a glioma antigen gene, comprising the following processes, (1) a process of constructing λ phage cDNA library comprising the steps of: total RNA is extracted and purified from a glioma cell line; cDNA is synthesized with the purified mRNA, and this cDNA is introduced into λ phage vector to infect E. coli, (2) a process of generating a reactive serum by eliminating reactants after applying the extract of E. coli to the diluted serum of a glioma patient, (3) a process of detecting positive clones which are reacted by an antibody in the serum by using a labeled anti-IgG antibody after applying the aforementioned λ phage cDNA library to the reactive serum, (4) a process of confirming antibody-reactivity after repeating several screenings for the detected positive clones, (5) a process of a serum screening using the antigens isolated from the positive clones together with at least the sera of glioma patients and healthy persons (claim 1); a diagnostic drug for detecting glioma comprising an antibody which specifically binds to whole or part of one or more types of glioma antigens obtained by the method for preparing glioma antigens and/or glioma antigen genes described in claim 1, and/or to whole or part of said glioma antigens (claim 2); a detecting and diagnostic probe for glioma comprising whole or part of antisense strand of DNA or RNA encoding one or more types of glioma antigens obtained by the method for preparing glioma antigens and/or glioma antigen genes described in claim 1 (claim 3); an anti-tumor agent with an antibody specifically binding to whole or part of one or more types of glioma antigens obtained by the method for preparing glioma antigens and/or a glioma antigen genes described in claim 1, and/or to whole or part of said glioma antigens as an effective ingredient (claim 4).

The present invention further relates to DNA encoding the following protein (a) or (b), (a) a protein comprising the amino acid sequence shown by SEQ ID NO:2, (b) a protein which comprises an amino acid sequence wherein one or a few amino acids are deleted, substituted or added in the amino acid sequence shown by SEQ ID NO:2, and which has the immunity-inducing activity (claim 5), DNA comprising the base sequence shown by SEQ ID No:1 or its complementary sequence and part or whole of these sequences (claim 6); DNA which hybridizes under a stringent condition with DNA of claim 6, and which encodes a protein having the immunity-inducing activity (claim 7); DNA encoding the following protein (a) or (b), (a) a protein comprising the amino acid sequence shown by SEQ ID NO:4, 6, 8, or 10, (b) a protein which comprises an amino acid sequence wherein one or a few amino acids are deleted, substituted or added in the amino acid sequence shown by SEQ ID NO:4, 6, 8, or 10, and which has the immunity-inducing activity (claim 8), DNA comprising the base sequence shown by SEQ ID No:3, 5, 7, or 9, or its complementary sequence and part or whole of these sequences (claim 9); DNA which hybridizes under a stringent condition with DNA of claim 9, and which encodes a protein having the immunity-inducing activity (claim 10); DNA encoding the following protein (a) or (b), (a) a protein comprising the amino acid sequence shown by SEQ ID NO:12, (b) a protein which comprises an amino acid sequence wherein one or a few amino acids are deleted, substituted or added in the amino acid sequence shown by SEQ ID NO:12, and which has the immunity-inducing activity (claim 11), DNA comprising the base sequence shown by SEQ ID No:11, or its complementary sequence and part or whole of these sequences (claim 12); DNA which hybridizes under a stringent condition with DNA of claim 12, and which encodes a protein having the immunity-inducing activity (claim 13).

The present invention still further relates to: a protein comprising the amino acid sequence shown by SEQ ID NO:2 (claim 14); a protein which comprises an amino acid sequence wherein one or a few amino acids are deleted, substituted or added in the amino acid sequence shown by SEQ ID NO:2, and which has the immunity-inducing activity (claim 15); a protein comprising the amino acid sequence shown by SEQ ID NO:4, 6, 8, or 10 (claim 16); and a protein which comprises an amino acid sequence wherein one or a few amino acids are deleted, substituted or added in the amino acid sequence shown by SEQ ID NO: 4, 6, 8, or 10, and which has the immunity-inducing activity (claim 17); a protein comprising the amino acid sequence shown by SEQ ID NO:12 (claim 18); a protein which comprises an amino acid sequence wherein one or a few amino acids are deleted, substituted or added in the amino acid sequence shown by SEQ ID NO:12, and which has the immunity-inducing activity (claim 19); a peptide comprising a partial protein of any of claims 14 to 19, and which has the immunity-inducing activity (claim 20).

The present invention also relates to: a fusion protein or a fusion peptide wherein the protein of any of claims 14 to 19 or the peptide of claim 20, and a marker protein and/or a peptide tag, are bound (claim 21); an antibody against the protein of any of claims 14 to 19 or the peptide of claim 20 (claim 22); a host cell comprising an expression system capable of expressing the protein of any of claims 14 to 19 or the peptide of claim 20 (claim 23); a non-human animal whose gene function to encode the protein of any of claims 14 to 19 or the peptide of claim 20 is deficient on its chromosome or which over-expresses the protein of any of claims 14 to 19 or the peptide of claim 20 (claim 24); a screening method for a promoter or a suppressor for the immunity-inducing activity wherein the immunity-inducing activity in T cell is measured and assessed by using the protein of any of claims 14 to 19, the peptide of claim 20, a test substance, and T cell (claim 25).

### BRIEF DESCRIPTION OF DRAWINGS

FIG. 1 is a drawing showing the result of expression analysis of glioma antigen KU-GB-1 by Northern blot method.

FIG. 2 is a drawing showing the result of expression analysis of glioma antigen KU-GB-2 by RT-PCR.

FIG. 3 is a drawing showing the result of expression analysis of glioma antigen KU-GB-5 by RT-PCR.

### BEST MODE OF CARRYING OUT THE INVENTION

A method for preparing a glioma antigen and/or a glioma antigen gene of the present invention is not particularly restricted as long as it includes the following processes, (1) a process of constructing λ phage cDNA library comprising the steps of: total RNA is extracted and purified from a glioma cell line; cDNA is synthesized with the purified mRNA, and this cDNA is introduced into λ phage vector to infect E. coli, (2) a process of generating a reactive serum by eliminating reactants after applying the extract of E. coli to the diluted serum of a glioma patient, (3) a process of detecting positive clones which are reacted by an antibody in the serum by using a labeled anti-IgG antibody after applying the aforementioned λ phage cDNA library to the reactive serum, (4) a process of confirming antibody-reactivity after repeating several screenings for the detected positive clones, (5) a process of a serum screening using the antigens isolated from the positive clones together with at least the sera of glioma patients and healthy persons. However, it is preferable to amplify cDNA insert of the positive clone obtained by the process (4) by PCR, and to determine its sequence, and then to conduct homology search with the use of the existing gene data base. Besides, it is preferable to put serum screening in the process (5) not only on the serum of glioma patients and healthy persons, but also on the serum of other brain disease and other cancer patients.

By way of the aforementioned preparation methods, two groups of antigens i.e. a group of glioma specific antigens (glioma specific antigen genes) reacting only to the sera of glioma patients, and a group of glioma non-specific antigens (glioma non-specific antigen genes) which react to the sera of both glioma and other cancer patients can be obtained. The group of glioma specific antigens (glioma specific antigen genes) is exemplified by novel KU-GB-2, KU-GB-5, L13 together with Cytochrome-C, DEK oncogene, Nuclear Antigen SP100, Sarcolemmal associated protein, KIAA1014 protein, MutL (E. coli) homolog1 (hMLH1) (GenBank Accession No. NM_{—}000249) etc. On the other hand, the group of glioma non-specific antigens (glioma non-specific antigen genes) is exemplified by KU-GB-1, Mitotic centromere associated protein, human S5A (GenBank Accession No.NM_{—}002810, U24704, U51007), or human pUb-R5 (GenBank Accession No.AB033605) as an isoform of human S5A, Minichromosome maintenance deficient-3, etc. Besides, by expressing these glioma antigens either in vivo or in vitro in dendritic cells that are strong antigen-presenting cells, immunity can be induced by injecting these antigen-expressing dendritic cells.

As for a diagnostic drug for detecting glioma of the present invention, a reagent comprising whole or part of one or more types of glioma antigens obtained by the aforementioned preparation method can be used. Part of glioma antigens can be exemplified by an antigen recognition site and the like of glioma antigens. Or when used in combination of more than one antigen, it is preferable to select from the aforementioned glioma specific antigen group or glioma non-specific group. Besides, as for a diagnostic drug for detecting glioma of the present invention, reagents comprising antibodies such as monoclonal antibodies specifically binding to whole or part of the aforementioned glioma antigens and the like can be used. When said antibodies are used in combination of more than one type, it is preferable to select from the antibodies against the aforementioned glioma specific antigen group or the glioma non-specific antigen group. Examples of diagnostic methods for glioma by using a diagnostic drug for detecting glioma of the present invention include: a method of investigating whether antibody reaction can be acknowledged by applying labeled glioma antigens to an IgG antibody of the serum obtained from a specimen; a method of image diagnosis of glioma of which metes and bounds are indefinite by administering into blood labeled antibodies such as fluorescence or Fe to glioma antigens.

There is no particular limitation to a detecting and diagnostic probe for detecting glioma of the present invention, as long as such reagent includes whole or part of antisense strand of DNA or RNA encoding one or more types of glioma antigens obtained by the aforementioned method for preparing glioma antigen genes. However, DNA or RNA comprising more than 20 bp is preferable, and it is also preferable to select from antisense strands of DNA or RNA encoding the aforementioned glioma specific antigen group or glioma non-specific antigen group when used in combination of more than one type of probes. A detecting and diagnostic method of glioma using a detecting and diagnostic probe for glioma of the present invention is exemplified by a method of detecting mRNA of the present glioma antigens obtained from a specimen by using a labeling antisense strands. The specific examples of test samples used for detection and diagnosis are genomic DNA, RNA or cDNA that are obtained from the cells of subjects, for example, biopsy such as blood, urine, saliva, tissues, etc., but the test samples will not be limited to these examples.

As for an anti-tumor agent of the present invention, a formulation comprising whole or part of one or more types of glioma antigens obtained from the aforementioned method for preparing glioma antigens as an effective ingredient can be used. Part of glioma antigen can be exemplified by an antigen recognition site of glioma antigen. And it is preferable to select from the aforementioned glioma specific antigen group or glioma non-specific antigen group when used in combination of more than one type of antigens. A formulation comprising antibodies such as monoclonal antibodies which specifically bind to whole or part of said glioma antigen as an effective ingredient can also be used for anti-tumor agent of the present invention. It is also preferable to select from the antibodies to the aforementioned glioma specific antigen group or glioma non-specific antigen group when more than one type of antibodies are used in combination. When the anti-tumor agents of the present invention are injected orally, intravenously, intradermally, sucutaneously, etc., an anti-tumor effect can be expected due to the increase in the T cell-inducing activity in vivo. Further, T cell can be induced into activated T cell by being stimulated in vitro by using anti-tumor agent of the present invention. For example, tumor reactive activated T cell will be induced when peripheral blood lymphocyte or tumor-infiltrating lymphocyte are stimulated by an anti-tumor agent of the present invention with IL-2, and said activated T cell can be effectively used for adoptive immunotherapy. Genetic therapy wherein genes are selectively introduced into glioma or missile therapy wherein anti-tumor agent is selectively made to act on glioma can be possible by using antibody such as monoclonal antibody of the present invention.

Proteins as objects of the present invention can be exemplified by novel antigen proteins or their isoforms among the glioma antigens obtained by the aforementioned preparation method of glioma antigens. Examples include: glioma KU-GB-1 comprising the amino acid sequence shown by SEQ ID NO:2; four isoforms of glioma KU-GB-2 i.e. glioma KU-GB-2a comprising the amino acid sequence shown by SEQ ID NO:4; glioma KU-GB-2b comprising the amino acid sequence shown by SEQ ID NO: 6; glioma KU-GB-2c comprising the amino acid sequence shown by SEQ ID NO:8; glioma KU-GB-2d comprising the amino acid sequence shown by SEQ ID NO:10; glioma KU-GB-5 comprising the amino acid sequence shown by SEQ ID NO:12; and a protein which comprises an the amino acid sequence wherein one or a few amino acids are deleted, substituted or added in the amino acid sequence shown by SEQ ID NO:2, 4, 6, 8, 10, or 12 and which has the immunity-inducing activity. The word immunity-inducing activity used herein stands for the activity inducing immunoreaction such as antibody production, cellular immunity, immunological tolerance, and among said immunity-inducing activity, the ones with the T cell inducing activity which increases the frequency of cytotoxic T cell (CTL) precursor cell in peripheral blood is particularly preferable. A peptide of the present invention can be exemplified by a peptide comprising part of said protein and, which has an immunity-inducing activity. Said peptide is not specifically restricted, but can be eligibly exemplified by a peptide comprising recognition site of antibodies, and a recognition site of CD4+ T cell and/or CD8+ T cell. In addition to the aforementioned proteins and peptides as objects of the present invention, recombinant proteins or peptides which specifically bind to antibodies of said proteins and peptides are also included in the present invention, which may collectively be referred to as “the present glioma antigen”. Besides, the origin of the present glioma antigen is not limited to human, and the present glioma antigen is useful for a diagnostic drug for detecting tumor or a screening method for a promoter or a suppressor for the immunity-inducing activity as described hereinafter.

DNA as an object of the present invention is exemplified by DNA encoding novel antigen proteins among the glioma antigens obtained from the aforementioned method for preparing glioma antigens. The specific examples include: DNA encoding the protein KU-GB-1 comprising the amino acid sequence shown by SEQ ID NO:2; DNA encoding protein KU-GB-2a comprising the amino acid sequence shown by SEQ ID NO:4; DNA encoding protein KU-GB-2b comprising the amino acid sequence shown by SEQ ID NO:6; DNA encoding protein KU-GB-2c comprising the amino acid sequence shown by SEQ ID NO:8; DNA encoding protein KU-GB-2d comprising the amino acid sequence shown by SEQ ID NO:10; DNA encoding protein KU-GB-5 comprising the amino acid sequence shown by SEQ ID NO:12; and DNA encoding a protein which comprises an amino acid sequence wherein one or a few amino acids are deleted, substituted or added in the amino acid sequence shown by SEQ ID NO:2, 4, 6, 8, 10, or 12, and which has the immunity-inducing activity, and DNA comprising the base sequence shown by SEQ ID No:1, 3, 5, 7, 9, or 11, or its complementary sequence and part or whole of these sequences.

Further, DNA encoding a protein of the interest, which has the same effect as glioma antigens such as KU-GB-1, KU-GB-2a, KU-GB-2b, KU-GB-2c, KU-GB-2d, KU-GB-5 etc., having the immunity-inducing activity can be obtained by the hybridization with a DNA library derived from various glioma cells under a stringent condition by using the base sequence shown by SEQ ID NO:1, 3, 5, 7, 9, or 11 or its complimentary sequence and part or whole of these sequences as a probe, and then by the isolation of DNA which hybridize with the probe. DNA obtained hereby is also within the scope of the present invention. A hybridization condition for obtaining the DNA of the present invention is exemplified by hybridization at 42° C., and washing at 42° C. in a buffer solution containing 1×SSC, 0.1% SDS, and more preferable exemplification is hybridization at 65° C. and washing at 65° C. in a washing buffer solution containing 0.1×SSC, 0.1% SDS. There are a number of factors other than the temperature condition mentioned above that affect the hybridization stringency and those skilled in the art can actualize the same stringency as that of the hybridization referred to in the above by appropriately combining various factors.

Besides, a gene or DNA encoding the present glioma antigen mentioned above, and the present glioma antigen and the like can be used for generating a fusion peptide or a fusion protein wherein the present glioma antigen and a marker protein and/or a peptide tag are bound, an antibody against the present glioma antigen, a host cell comprising expression system capable of expressing the present glioma antigen, a non-human animal whose gene function to encode the present glioma antigens is deficient on its chromosome, or a non-human animal over-expressing the present glioma antigen as specifically described hereinafter.

Any fusion protein and fusion peptide may be used as a fusion protein and a fusion peptide for the present invention as long as the present glioma antigens bind to marker proteins and/or peptide tags. As for a marker protein, the specific examples include conventionally known ones such as alkaline phosphatase, the Fc region of an antibody, HRP, GFP, etc. Conventionally known peptide tags including His tag, FLAG tag, S tag, etc. are the specific examples of the peptide tags for the use in the present invention. However, there is no limitation to these examples. These fusion proteins can be generated according to the ordinary protocols and are useful for the following: purification of the glioma antigen KU-GB-1, KU-GB-2a, KU-GB-2b, KU-GB-2c, KU-GB-2d, KU-GB-5 or the like using affinity of Ni-NTA and a His tag; detection of a protein having the T cell-inducing activity; quantification of an antibody against the glioma antigen KU-GB-1, KU-GB-2a, KU-GB-2b, KU-GB-2c, KU-GB-2d, KU-GB-5 or the like; as a diagnostic marker for cancer such as glioma and the like; as a laboratory reagent in this field of art.

An antibody against the present glioma antigen of the present invention can be particularly exemplified by an immune-specific antibody such as a monoclonal antibody, a polyclonal antibody, a chimeric antibody, a single-stranded antibody, a humanized antibody, etc. These antibodies can be generated according to the ordinary protocols by using the present glioma antigen. However, an monoclonal antibody is more preferable than the other sorts of antibodies mentioned above because of its specificity, and particularly, monoclonal antibodies specifically recognizing epitopes such as KU-GB-1, KU-GB-2a, KU-GB-2b, KU-GB-2c, KU-GB-2d, KU-GB-5 etc., or the complex of epitopes and HLA are more preferable. Such monoclonal antibodies or the like are useful not only for diagnosis of cancers such as glioma and missile therapy, but also for elucidating the development mechanism of malignant tumors such as glioma and the like.

Antibodies of the present invention can be created by administering to an animal (preferably non-human) the present glioma antigen, their fragments containing epitopes, or cells expressing the present glioma antigens, particularly an complex of epitopes and HLA, on the membrane surface, according to the conventional protocols. The monoclonal antibodies can be prepared, for instance, by any optional method such as a hybridoma method that brings antibodies produced by cultured materials of continuous cell line (Nature 256, 495-497, 1975), a trioma method, a human B-cell hybridoma method (Immunology Today 4, 72, 1983), and an EBV-hybridoma method (MONOCLONAL ANTIBODIES AND CANCER THERAPY, pp.77-96, Alan R. Liss, Inc., 1985), etc.

The method for preparing a single chain antibody (U.S. Pat. No. 4,946,778) can be adopted to prepare single-stranded antibodies to the present glioma antigens of the present invention mentioned above. Besides, transgenic mice, other mammals, etc. can be used for expressing humanized antibodies, clones expressing the present glioma antigens can be isolated/identified using the antibodies mentioned above, and their polypeptides can be purified by affinity chromatography. Antibodies to the present glioma antigens or to their peptides containing antigenic epitopes can possibly be used for diagnosis and therapy for glioma etc. Furthermore, recombinant proteins or peptides to which these antibodies specifically bind are also covered by the present glioma antigens of the present invention as described earlier.

A host cell comprising an expression system capable of expressing the present glioma antigens can be constructed by introducing genes encoding the present glioma antigens into the host cell, by the methods described in many standard laboratory manuals such as manuals of Davis et al. (BASIC METHODS IN MOLECULAR BIOLOGY, 1986) and of Sambrook et al. (MOLECULAR CLONING: A LABORATORY MANUAL, 2nd Ed., Cold Spring Harbor Laboratory Press, Cold Spring Harbor, N.Y., 1989), and the specific examples include calcium-phosphate transfection, DEAE-dextran-mediated transfection, transvection, microinjection, cationic lipid-mediated transfection, electroporation, transduction, scrape loading, ballistic introduction, infection, etc.

The examples of host cells mentioned above include bacterial prokaryotic cells such as E. coli, Streptomyces, Bacillus Subtilis, Streptococcus, Staphylococcus, etc., eukaryotic cells such as yeast, aspergillus, etc., insect cells such as Drosophila S2, Spodoptera Sf9, etc., animal cells such as L cell, CHO cell, COS cell, HeLa cell, C127 cell, BALB/c3T3 cell (including mutants deficient in dihydrofolate reductase, tymidine kinase, etc.), BHK21 cell, HEK293 cell, and plant cells or the like. Further, a host cell having a HLA-expressing ability, and which comprises an expression system capable of expressing the present glioma antigen can be generated by introducing a gene encoding the present glioma antigen into a host cell having a HLA-expressing ability or a host cell not originally having said ability to those which HLAcDNA was transfected according to the aforementioned methods.

There is no limitation to an expression system as long as the expression system is capable of expressing the present glioma antigens described above in a host cell and the examples include chromosome-, episome- and virus-derived expression systems, for instance, vectors derived from bacterial plasmid, vectors derived yeast plasmid, vectors derived papovavirus such as SV40, vaccinia virus, adenovirus, adeno-associated virus, chicken pox virus, pseudorabies virus, retrovirus, and vectors derived bacteriophage or transposon and vectors derived the combination of these two, e.g. vectors derived from genetic factors of plasmid and bacteriophage, such as cosmid and phagemid. The expression systems may comprise a control sequence for regulating the expression in addition to a sequence for raising the expression.

Host cells comprising the above-mentioned expression systems and the cell membranes of the cells, or the present glioma antigens obtained by culturing the cells can be used in the screening methods of the present invention as described below. For example, the method of F. Pietri-Rouxel et al. (Eur. J. Biochem., 247, 1174-1179, 1997) or the like can be used to obtain cell membranes. Further, to collect and purify the present glioma antigens from the cell culture, the known methods can be adopted including ammonium sulfate- or ethanol-precipitation, acid extraction, anion- or cation-exchange chromatography, phosphocellulose chromatography, hydrophobic interaction chromatography, affinity chromatography, hydroxyapatite chromatography and lectin chromatography, where the high performance liquid chromatography is preferably used. As a column especially used for affinity chromatography, columns to which antibodies to the present glioma antigens are bound, for instance, are used, and when ordinary peptide tags are added to the present glioma antigens mentioned above, columns to which substances having affinity with the peptide tags are bound are used in order to obtain the present glioma antigens.

A non-human animal whose gene function to encode the present glioma antigens of the present invention is deficient on its chromosome means a non-human animal part or whole of whose gene on its chromosome encoding the present glioma antigens is inactivated by gene mutations such as disruption, deletion, replacement, etc., so that whose function to express the present glioma antigens is lost. Further, a non-human animal which over-expresses the present glioma antigens is specifically exemplified by a non-human animal which produces larger amount of the present glioma antigens than a wild-type non-human animal does. Although rodents or the like such as mice, rats, etc. are particularly exemplified for non-human animals of the present invention, the examples will not be limited to these animals only.

Homozygous non-human animals that are born according to Mendel's Law include the deficient type or the over-expressing type for the present glioma antigens as well as their wild type littermates. By using the deficient type animals or the over-expressing type animals of these homozygous non-human animals together with their wild-type littermates at the same time, accurate comparative experiments can be carried out on the individual level. In performing screening of the present invention described below, it is, therefore, preferable to use the wild type non-human animals, i.e. animals of the same species as, or even better the littermates of, non-human animals whose gene function to encode the present glioma antigens is deficient or over-expressing on their chromosomes in parallel with the deficient or over-expressing type animals. The method of producing a non-human animal whose gene function to encode the present glioma antigens is deficient or over-expressing on its chromosome is now explained in the following with the present glioma antigen knockout mouse and the present glioma antigen transgenic mouse.

A mouse, for instance, whose gene function to encode the present glioma antigen is deficient on its chromosome i.e. the present glioma antigen knockout mouse is generated by the following steps. A gene encoding the present glioma antigen is screened by using a gene fragment obtained by a method such as PCR or the like from the mouse gene library. A screened gene which encodes the present glioma antigens is subcloned with a viral vector or the like and is identified by DNA sequencing. Then whole or part of a gene of this clone which encodes the present glioma antigen is substituted with a pMC1 neo gene cassette or the like. A gene such as a diphtheria toxin A fragment (DT-A) gene, a herpes simplex virus tymidine kinase (HSV-tk) gene, etc. is introduced onto the 3′-end, and thus a targeting vector is constructed.

The targeting vectors thus constructed are linearlized and introduced into ES cells by electroporation or the like to cause homologous recombination. Among the homologous recombinants, ES cells in which homologous recombination have occurred are selected by the use of antibiotics such as G418, ganciclovir (GANC), etc. It is preferable to confirm whether the selected ES cells are the recombinants of the interest by Southern blotting or the like. A clone of the ES cells confirmed is microinjected into a mouse blastocyst and which blastocyst is placed back to the recipient mouse to generate a chimeric mouse. A heterozygous mouse can be obtained by intercrossing the chimeric mouse and a wild type mouse. By further intercrossing the heterozygous mice, the present glioma antigen knockout mice of the present invention can be generated. Whether the present glioma antigens knockout mouse is generated is examined by Northern blotting upon isolating RNA from the mouse obtained by the above-described method and by Western blotting or the like with which the expression of the mouse can be directly examined.

The present glioma antigen transgenic mouse is created by the following steps. A promoter such as chicken β-actin, mouse neurofilament, SV40, etc. and poly (A) such as rabbit β-globin, SV40, etc. or introns are fused with cDNA encoding the present glioma antigen to generate a transgene. This transgene is microinjected into the pronucleus of a mouse fertilized egg. After the obtained egg cell is cultured, it is transplanted to the oviduct of the recipient mouse which was fed thereafter. Neonetal mice that have the aforementioned cDNA were selected from among all the mice born and thus the transgenic mice are created. Neonatal mice having the cDNA can be selected by extracting crude DNA from the mice tails or the like and then by a dot hybridization method using a gene encoding the introduced present glioma antigen as a probe and by PCR method or the like using a specific primer.

As for a screening method for a promoter or a suppressor for the immunity-inducing activity of the present invention, it is not specifically restricted as long as the method comprises the measurement and assessment of the immunity-inducing activity of T cell by using the present glioma antigen, a test substance, and T cell. Examples include methods wherein immunity-inducing activity in said T cell is measured and assessed by using the following: a test substance, the present glioma antigen, and T cell; a test substance, a cell membrane or a cell which expresses the present glioma antigens, and T cell; T cell and a host cell expressing the present glioma antigens and to which vectors expressing HLA are transfected together with vectors expressing a test polypeptide; T cell and a host cell expressing the present glioma antigens which has a HLA-expressing ability and which is transfected with vectors expressing a test polypeptide, and a method wherein immunity-inducing activity in a T cell is measured and assessed on non-human animals by administering a test substance to said non-human animals such as the aforementioned knock-out mice and transgenic mice. Specific examples of the aforementioned cell membrane or a cell include: the primarily cultured cells or the like obtained from non-human animals wherein a gene encoding the present glioma antigen is over-expressing, or wild type of non-human animals; a host cell comprising an expression system capable of expressing the aforementioned present glioma antigens; cell membrane of these cells. The method of contacting a cell membrane or cell and a test substance is exemplified by a method wherein a cell membrane or a cell which expresses the present glioma antigen is cultured in vitro under the presence of a test substance, and subsequently contacted with T cell. A method wherein immunity-inducing activity in a T cell is measured and assessed can be specifically exemplified by a method of measuring which has an amount of IFN r released from T cell onto culture media as an indicator. Further, a promoter for the immunity-inducing activity obtained by the aforementioned screening method can be applied to the therapy for patients in need of the promotion of immunity-inducing activity. Meanwhile, a suppressor for the immunity-inducing activity can be applied to the therapy for patients in need of the suppression of the immunity-inducing activity.

The present invention will be further specifically explained in the following with reference to the examples, but the technical scope of the invention will not be limited to these examples.

### Cell Lines and Tissues

Human glioma cell line (GI-1, T98G, U87MG, U251), human malignant melanoma cell line (SKmel23, 1362mel, 888mel), lung cancer cell line (LU99, RERF-LC-MA, EBC1), esophageal cancer cell line (TE10), breast cancer cell line (MDA231), pancreatic cancer cell line (PK1), renal cell carcinoma cell line (RCC6), bladder carcinoma cell line (BC47), prostate cancer cell line (PC3), leukemia cell line (HL60, Molt4) are cultured in RPMI comprising 10% of a fetal bovine serum to which penicillin (100 IU/ml) and streptomycin (100 μg/ml) is added respectively. Pancreatic cancer cell line (KU7) was cultured in RPMI1640 comprising 10% of a fetal bovine serum to which penicillin/streptomycin (1%), L-glutamine (1%), HEPES (10 mM), EGF (6 μg/l), insulin (150 U/l), hydrocortisone (0.5 mg/l), transferrin (10 mg/l) are added. Part of lesion extirpated during an operation was applied to a human glioma tissue (GB13, GB16, GB17, GB4). Total RNA of normal tissues (brain, heart, lung, stomach, colon, liver, spleen, small intestine, renal, testis, placenta, muscle, embryonic brain) were purchased from Clontech.

### Generating of cDNA Library

Total RNA was isolated from the aforementioned glioma cell line U87-MG and T98G by the cecium chloride ultracentrifuge method. After mixing 1 mg of each cell line, mRNA was purified by way of poly (A) selection using oligotex (dT) 30 (TAKARA). cDNA was synthesized by reverse transcription by using ZAP-cDNA Synthesis Kit (Stratagene) from 5 μg of purified mRNA. A phage cDNA library was constructed by incorporating said cDNA into A ZAP expressing phagemid vector.

### Serum Screening of cDNA Library

The cDNA library constructed as above is sprayed onto 150 mm NZY agarose plate by 1×10⁴ clone, and cultured at 42° C. for 4 hours, and then at 37° C. for 4 hours. Thereafter, recombinant protein of which expression was induced by 10 mM of IPTG on E. coli (XL1-Bllue) was transcribed onto nitro cellulose filter (Hybond-c, Amersham, Buckinghamshire, England). After adsorbed bacteria or phage are removed by washing the filter with TBS comprising 0.05% of TWIN20 (10 mM Tris-HCl, 150 mM Nacl; pH7.5), non-specific reaction was suppressed by TBS comprising 5% of skimmed milk. This filter was applied to a patient's serum which had been diluted for 100 times or 400 times at 37° C. for 4 hours. As for the sera of patients, the ones collected from 12 glioma patients as shown in Table 1, and the ones collected from 8 glioma patients as shown in Table 2 and combined with the sera of 4 glioma patients [(the sera of the patients G20, G21, G22, G27 were combined), (the sera of the patients G18, G23, G24, G28 were combined)] were applied. These sera were preserved at −80° C., and diluted five times with TBS solution comprising 5% of skimmed milk immediately prior to be used. These sera were further combined with lysate of bacteriolysed E. coli at 1:2 and reacted at 4° C. for 8 hours in order to precedently remove the antibody which reacts to bacteria. Its supernatant was ultimately diluted for 100 times (as for the cases of sera of patients shown in Table 1 was used) or 400 times (as for the cases of sera of 4 patients shown in Table 2 was used.).

TABLE 1

| Patients' | | | | positive | different |
|---|---|---|---|---|---|
| serum | age | sex | diagnosis | clones | inserts |
| G | 48 | M | Glioblastoma | 5 | 4 |
| H | 77 | F | Anaplastic | 4 | 4 |
| | | | oligoastrocytoma | | |
| I | 53 | F | Glioblastoma | 3 | 2 |
| K | 26 | F | Glioblastoma | 18 | 8 |
| L | 61 | M | Anaplastic | 15 | 5 |
| | | | oligoastrocytoma | | |
| N | 32 | F | Anaplastic | 9 | 7 |
| | | | astrocytoma | | |
| O | 62 | F | Glioblastoma | 1 | 1 |
| P | 30 | M | Anaplastic | 6 | 3 |
| | | | astrocytoma | | |
| R | 53 | F | Glioblastoma | 18 | 12 |
| S | 49 | F | Astrocytoma | 8 | 2 |
| U | 61 | F | Glioblastoma | 1 | 1 |
| V | 68 | F | Glioblastoma | 4 | 3 |

| | | |
|---|---|---|
| total | 92 | 52 |

TABLE 2

| | | | | | | | types of |
|---|---|---|---|---|---|---|---|
| Patients | | | | | positive | | isolated |
| serum | age | sex | diagnosis | | clones | | genes |
| G20 | 17 | F | Anaplastic | | | | |
| | | | Astrocytoma | | | | |
| G21 | 32 | M | Astrocytoma | | | | |
| G22 | 18 | M | Astrocytoma | {close oversize bracket} | 4 | {close oversize bracket} | 3 |
| | | | Anaplastic | | | | |
| G27 | 25 | M | Astrocytoma | | | | |
| G18 | 21 | F | Glioblastoma | | | | 5 |
| G23 | 3 | F | Astrocytoma | | | | (incl. |
| G24 | 7 | F | Oligoastrocytoma | {close oversize bracket} | 17 | {close oversize bracket} | a |
| G28 | 41 | M | Anaplastic | | | | novel |
| | | | oligoastrocytoma | | | | type) |

| | | |
|---|---|---|
| Total | 21 | 8 |

Said processed serum was reacted to recombinant protein blotted onto nitro cellulose filter at 37° C. for 4 hours, and then recombinant protein which was reacted by antibodies within a serum was detected by using alkaline phosphatase-labeled anti-human IgG antibody (ICN Pharmaceuticals). Thereafter, positive clones which coincide with coloring reactive positive site was recovered from the aforementioned 150 mm NZY agarose plate and dissolved into SM buffering solution (100 mM of NaCl, 10 mM of MgSO₄, 50 mM of Tris-HCl, 0.01% of gelatin; ph7.5). This screening was further conducted for several times, and thus antibody reactivity of the positive clones was confirmed. As a result of the screening from 5×10⁵ phage clones per serum of one patient, 92 positive clones from the patients' sera shown in Table 1 and 21 positive clones from the patient' sera shown in Table 2 were isolated. Number of isolated positive clones by each patient are shown in Table 1 and 2. Among the 92 positive clones obtained from the patients' sera shown in Table 1, 18 clones were obtained from the patient K of Glioblastoma, 18 clones were from the patient R of Glioblastoma, and 15 clones were from the patient L of Anaplastic oligoastrocytoma. Herein a tendency wherein positive clones are concentrated to the specific patients' sera was acknowledged, however, this tendency appeared to have no apparent relationship with patients' age or malignancy.

### Homology Search of Isolated Antigen Gene

cDNA inserts which were integrated into 92 phages obtained by using the patients' sera shown in Table 1 and 21 phages obtained by using the patients' sera shown in Table 2 were amplified by PCR method and each base sequence was determined. ExTaq (TAKARA) was used as a reactive enzyme, T3 (5′-AATTAACCCTCACTAAAGGG-3′; SEQ ID NO:13) was used as a sense primer, and T7 (5′-GTAATACGACTCACTATAGGGC-3′ SEQ ID NO:14) was used as an anti-sense primer. Reaction condition was as follows: a cycle of denaturation for 5 minutes at 94° C. at the beginning only by using thermal cycler (Perkin Elmer), followed by thermal denaturation for one minute at 94° C., annealing for 1 minute at 55° C., and extension for 2 minutes at 72° C. was repeated 35 times, and extension for 7 minutes at 72° C. was conducted lastly. Approximately 300-500 bp of the base sequence on 5′ end were determined by DNA sequencing of the PCR products obtained herein with applications of Big Dye DNA Sequencing Kit (ABI) and ABI310 auto sequencer. The DNA determined herein was compared with gene information registered in the gene data base of National Center for Biotechnology Information or EST data base, and homology to the existing genes was searched. As a result, 92 positive clones obtained by using patients' sera shown in Table 1 were turned out to be 52 types of antigen cDNA comprising cDNA encoding 38 types of known proteins and cDNA encoding 14 types of new proteins. The number of 52 types of antigen cDNA present in each patient is shown in Table 1. Further, 21 positive clones obtained by using patients' sera shown in Table 2 were turned out to be 8 types of antigen cDNA comprising cDNA encoding 7 types of known proteins and cDNA encoding 1 type of new protein. The number of 8 types of antigen cDNA present in each patient is shown in Table 2.

### Serum Screening

In order to consider the application to a serum diagnosis of glioma by using each clone expressing 52 types of antigens isolated by using patients' sera as shown in Table 1, screening of each serum which was diluted for 100 times of the following was raised: 17 glioma patients (Glioblastoma 9, Anaplastic astrocytoma 5, Anaplastic oligoastrocytoma 2, diffuse Astrocytoma 1); for part of antigens, 29 glioma patients (Glioblastoma 12, Anaplastic astrocytoma 7, Anaplastic oligoastrocytoma 4, Astrocytoma 4, Oligoastrocytoma 2); 8 patients of other brain deseases (malignant lymphoma 2, brain abscess 1, meningioma 3, metastatic glioma 1, subarachnoid bleeding 1); 16 patients of other cancer (malignant melanoma 4, pancreatic cancer 4, renal cancer 4, esophageal cancer 4); and 16 healthy persons (26 persons or 54 persons for a part of antigens). It was investigated whether IgG antibody against each isolated glioma antigen can be detected in these sera. As a result, the following antigens were obtained: 7 types of glioma-specific antigens (glioma specific antigen genes) which react exclusively with the sera of glioma patients; 4 types of glioma non-specific antigens (glioma non-specific antigen genes) which react with the sera of glioma patients and patients of other cancers but do not react with sera of other healthy persons. 7 types of glioma specific antigens (glioma specific antigen genes) included Cytochrome-C, DEK oncogene, Nuclear Antigen SP100, Sarcolemmal associated protein, K1AA1014 protein in addition to the novel KU-GB-2, L13 as shown in Table 3. Besides, 4 types of glioma non-specific antigens (glioma non-specific antigen genes) included Mitotic centromere associated protein, human S5A, Minichromosome maintenance deficient-3, in addition to the novel KU-GB-1 as shown in Table 4. The sera of cancer patients other than glioma which react to said glioma non-specific antigens are as follow; melanoma and renal cancer patients' sera for KU-GB-1, pancreatic cancer patients' sera for Mitotic centromere associated protein, esophageal cancer patients' sera for human S5a, melanoma patients' sera for Minichromosome maintenance deficient-3.

TABLE 3

| | | | Patients | |
|---|---|---|---|---|
| | | | of | Patients |
| | | | Other | of |
| Antigens | Glioma | Healthy | Brain | other |
| (Antigen genes) | Patients | Persons | Diseases | cancers |
| KU-GB-2 | 5/29 | 0/54 | 0/8 | 0/16 |
| L13 | 2/17 | 0/16 | 0/8 | 0/16 |
| Cytochrome-C | 1/17 | 0/16 | 0/8 | 0/16 |
| DEK oncogene | 1/17 | 0/16 | 0/8 | 0/16 |
| Nuclear Antigen SP100 | 1/17 | 0/16 | 0/8 | 0/16 |
| Sarcolemmal | 1/17 | 0/16 | 0/8 | 0/16 |
| associated protein | | | | |
| KIAA1014 protein | 1/17 | 0/16 | 0/8 | 0/16 |

TABLE 4

| | | | Patients | |
|---|---|---|---|---|
| | | | of | Patients |
| | | | Other | of |
| Antigens | Glioma | Healthy | Brain | other |
| (Antigen genes) | Patients | Persons | Diseases | cancers |
| KU-GB-1 | 1/29 | 0/54 | 0/8 | 2/16 |
| Mitotic centromere | 2/17 | 0/16 | 0/8 | 1/16 |
| associated protein | | | | |
| human S5a | 3/17 | 0/26 | 0/8 | 1/16 |
| Minichromosome | 1/17 | 0/16 | 0/4 | 1/16 |
| maintenance deficient-3 | | | | |

The aforementioned 7 types of glioma specific antigens and 4 types of glioma non-specific antigens are considered to be effective as serum diagnosis products or therapy products for glioma or cancer including glioma, since they do not show an antibody reaction against the sera of other brain disease patients or healthy persons. For example, as in the result of detecting the aforementioned IgG antibody, antibody reaction against human S5a was found in the sera of glioma patients and esophageal cancer patients, but no reaction was found in the sera of other brain disease patients and healthy persons. Therefore, human S5a is considered to be effective as serum diagnosis products or therapy products for cancers including glioma. In addition, this human S5a is registered by 3 types of the names to GenBank [Human proteasome 26S subunit, non-ATPase 4 (PSMD4): Accession No.NM_{—}002810, Antisecretory factory 1: Accession No.U24704, 26S protease subunit S5a:Accession No.U51007]. It is reported (EMBO J.19, 4144-4153, 2000) that one type of isoform showing neuro-specific expression is present in this human S5a. There is a high possibility that this isoform i.e. human pUb-R5 (Accession NO.AB033605) is also a glioma antigen as human S5a.

In order to consider the application to serum diagnosis of glioma by using each clone expressing 8 types of antigens isolated by using patients' sera as shown in Table 2, screenings for each serum diluted for 100 times of the following were raised: 29 glioma patients (Glioblastoma 12, Anaplastic astrocytoma 7, Anaplastic oligoastrocytoma 4, Astrocytoma 4, Oligoastrocytoma 2); 14 patients of other brain disease (malignant lymphoma 2, brain abscess 1, subarachnoid bleeding 1, Parkinson's disease 2, meningioma 4, neurocytoma 1, craniopharyngioma 1, metastatic brain tumor 2); and 37 healthy persons. It was investigated whether IgG antibody against each isolated antigen can be detected in these sera in a manner similar to the aforementioned. As a result, 2 types of glioma-specific antigens (glioma specific antigen genes) were found, one of which reacts exclusively with the sera of glioma patients, and the other one of which reacts with most of the sera of glioma patients but showed little reaction with the sera of healthy persons. These 2 types of glioma specific antigens (glioma specific antigen genes) were found to be a novel KU-GB-5 and MutL as shown in Table 5 (E. coli) homolog1 (hMLH1) (GenBank Accession No.NM_{—}000249). The aforementioned 2 types of glioma-specific antigens are considered to be effective as glioma serum diagnosis products or therapy products because these antigens react specifically with the sera of glioma patients or with the sera of most of glioma patients.

TABLE 5

| | | Serum Screening | | |
|---|---|---|---|---|
| | GeneBank | | | Patients of |
| | Accession | Glioma | Healthy | Other Brain |
| Genes | NO. | Patients | Persons | Disease |
| KU-GB-5 | None | 1/29 | 0/37 | 0/14 |
| MutL | NM000249 | 5/29 | 1/37 | 0/14 |
| (E. coli) | | | | |
| homolog1 | | | | |
| (hMLH1) | | | | |

### Determination of Base Sequence and the Amino Acid Sequence of Antigens

As a result of investigating the base sequence of isolated clones by using the automatic DNA sequencer (ALF Express, Pharmacia Biotech), it was found that KU-GB-1 consisted of the base sequence (SEQ ID NO:1) with the full length of 3709 bp comprising the genes encoding 1120 amino acid sequences (SEQ ID NO: 2). On the contrary, it was found that 4 types of isoforms are present in KU-GB-2. Namely, KU-GB-2a which consists of the base sequence (SEQ ID NO: 3) with the full length of 3533 bp comprising the genes encoding 1154 amino acid sequences (SEQ ID NO:4), together with 3 types of isoforms i.e. KU-GB-2b, KU-GB-2c, and Ku-GB-2d were found. The base sequence and the amino acid sequence of KU-GB-2b are shown respectively in SEQ ID NOS:5 and 6, the base sequence and the amino acid sequence of KU-GB-2c are shown respectively in SEQ ID NOS:7 and 8, the base sequence and the amino acid sequence of KU-GB-2d are shown respectively in SEQ ID NO:9 and 10. It was further found that KU-GB-5 consists of the base sequence (SEQ ID NO:11) with the full length of 3847 bp comprising the genes encoding 891 amino acid sequences (SEQ ID NO:12).

### Northern Blot Method for Detecting the mRNA Expression of Glioma Antigens

Northern Blot was conducted for KU-GB-1 by using RNA derived from normal tissues (brain, heart, kidney, spleen, liver, small intestine, lung, testis, placenta, stomach, RNA derived from human glioma cell line (GI-1, U87-MG, T98G), or human glioma tissues (GB13, GB17), RNA derived from the following cell lines: human malignant melanoma cell line (1362mel, 888mel); lung cancer cell line (LU99, RERF-LC-MA); esophageal cancer cell line (TE10); breast cancer cell line (MDA231); pancreatic cancer cell line (PK1); renal cell carcinoma cell line (RCC6); bladder carcinoma cell line (BC47); prostate cancer cell line (PC3); and leukemia cell line (HL60, Molt4). RNA (11 μg each) was electrophoresed in agarose gel comprising formamid NOe and formaldehyde, and removed into nylon membrane filter (Hybond XL, Amersham).

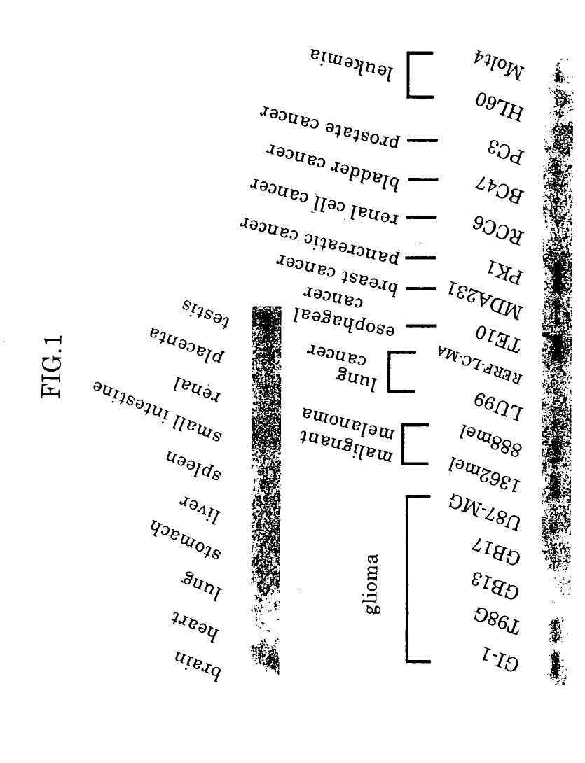

Secondly, a gene fragment comprising KU-GB-1 with the full length of 3709 bp was labeled with ³²P as a probe by using High Prime DNA Labeling Kit (Boehringer). Nylon membrane filter was soaked in Quick Hyb (Stratagene) which is used as hyblidization buffer. Pre-hyblidization was conducted at 68° C. for 30 minutes. Thereafter, hyblidization was conducted at 68° C. for 2 hours in the Quick hyb to which the aforementioned probe was added. And then, said hyblidized nylon membrane filter was washed with the lavage fluid I (2×SSC, 0.1% of SDS) at the room temperature for 15 minutes for two times, and then with the lavage fluid II (0.1×SSC, 0.1% of SDS) at 68° C. for 15 minutes for two times. Detecting for radioactive signal was conducted with the washed nylon membrane filter by using Molecular Imager (BIORAD), of which result is shown in FIG. 1.

As shown in FIG. 1, gene expression of KU-GB-1 in the normal tissues was not found except in testis, but high-level expression was found only in the cancer cell lines including glioma. Therefore, KU-GB-1 gene is considered to be effective as genetic diagnosis products for cancer including glioma. Further, as described above, having considered the fact that antibody reaction against KU-GB-1 antigens was acknowledged with the sera of glioma and other cancer (malignant melanoma, renal cancer) patients, but no antibody reaction against other brain disease patients or healthy persons was acknowledged, KU-GB-1 antigens are considered to be effective as diagnostic drugs or therapy products for cancer including glioma. Regarding the expression in testis, there is considered to be little possibility that a damage of testis could be a problem when KU-GB-1 antigen is practically used for immunotherapy and the like since this tissue is insulated from immune-system.

### RT-PCR Method for Detecting the Expression of Glioma Antigens

Subsequently, the expression specificity of glioma antigens KU-GB-2 genes or KU-GB-5 genes were investigated by the RT-PCR method in each normal tissue, glioma cell lines and tissues, and other tumor cell lines. A panel of cDNA as a template of RT-PCR was generated in total reaction amount of 200μl at 42° C. by using each 5 μg of RNA derived from the following; normal tissues such as brain, heart, lung, stomach, liver, pancrea, small intestine, kidney, placenta, testis, colon, muscle, embryonic brain (purchased exclusively from Clontech), 4 types of glioma cell lines (GI-1, U251, U87MG, and T98G), 4 types of glioma tissues (GB13, GB16, GB17, GB4), cell lines such as malignant melanoma cell line (Skmel23), lung cancer cell line (LU99, EBC1), esophageal cancer cell line (TE10), pancreatic cancer cell line (KU7), breast cancer cell line (MDA231), and leukemia cell line (Molt4), and a avian myeloblastosis virus reverse transcriptase (TAKARA) and oligo (dT) as a primer.

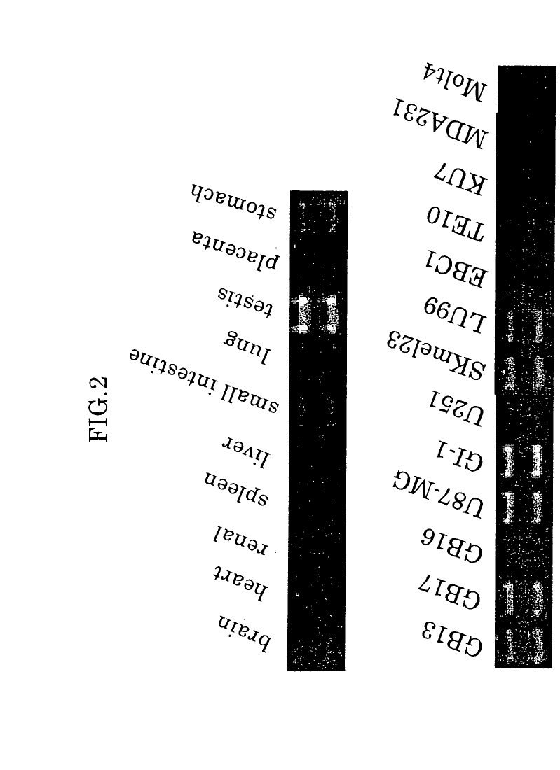

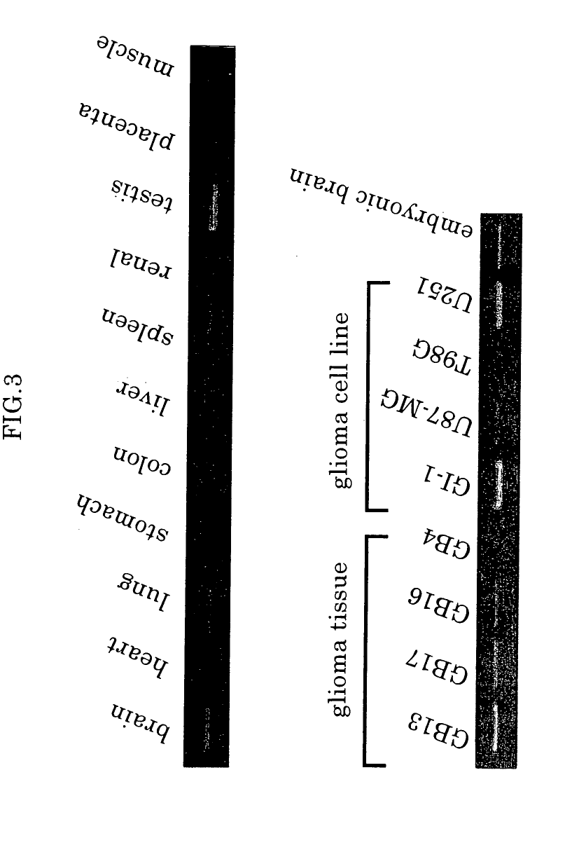

For detecting KU-GB-2, (5′-AGAGCGACCTTGAGACCAGAAA-3′; SEQ ID NO:15) was used as a sense primer, and (5′-ACACAAGGCAGGAGATGGAAGT-3′; SEQ ID NO:16) was used as an anti-sense primer. For detecting KU-GB-5, (5′-AAAACTCGCCTTTCTGAACC-3′; SEQ ID NO:17) was used as a sense primer, and (5′-AGCAAGTAACTGGAGAAGCA-3′; SEQ ID NO: 18) was used as an anti-sense primer. For detecting β actin (c551 bp) as a control, (5′-GTCGACAACGCATCCGGCATGTGCA-3′; SEQ ID NO:19) was used as a sense primer, and (5′-GGATCTTCATGAGGTAGTCAGTCAG-3′; SEQ ID NO:20) was used as an anti-sense primer. A cycle wherein a thermal denaturation is conducted at 94° C. for one minute by using these PCR primers, EXTaq (TAKARA) as a reactive enzyme, and thermal cycler (Perkin-Elmer), and stretch reacting is held at 72° C. for one minute after annealing was repeated for 30 times. Annealing temperature was set at Tm of the primers [62° C. for KU-GB-2, 60° C. for KU-GB-5, 68° C. for β actin as a control], and conducted for 1 minute (30 seconds for KU-GB-5). PCR products obtained herein was electrophoresed in agarose gel (2.0%), and stained by ethidium bromide (EtBr), and then band was detected by ultraviolet irradiation. A result of RT-PCR for KU-GB-2 is shown in FIG. 2, and a result of RT-PCR for KU-GB-5 is shown in FIG. 3 respectively. As shown in FIG. 2, strong expression of KU-GB-2 was found in U87-MG, GI-1 (human glioma cell line), GB13, GB17 (glioma tissue), Skmel23 (malignant melanoma cell line), and testis. Expression was weak in U251 (human glioma cell line), LU99 (lung cancer cell line), brain, small intestine, and stomach. Further, two bands are acknowledged in FIG. 2. But as mentioned above, 4 types of isoforms are present in KU-GB-2, of which bigger bands (top lane; 628 bp) show the expression of KU-GB-2b and KU-GB-2d, and smaller bands (bottom lane; 4476 bp) show the expression of KU-GB-2a and KU-GB-2c. And FIG. 3 shows that the strong expression of KU-GB-5 was acknowledged in GI-1, U251 (human glioma cell line), GB13, GB17 (glioma cell line), and testis, and weak expression can be seen in U87MG (human glioma cell line), GB16 (glioma tissue), brain, lung, spleen, and embryonic brain.

As shown in FIGS. 2 and 3, the high-level expression of genes cannot be seen in the normal tissues except for testis but only in glioma or Skmel 23 (malignant melanoma cell line) with KU-GB-2 and KU-GB-5 as is the case with KU-GB-1. Further, RT-PCR analysis was conducted respectively for 4 types of isoform which are present in KU-GB-2 as aforementioned, and as a result, it appeared that KU-GB-2a and KU-GB-2b show a expression pattern which is specific to nervous systems. Judging from these factors, individual genes belong to KU-GB-1 gene are also considered to be effective as genetic diagnosis products for cancer including glioma. Further, as described above, having considered the fact that antibody reaction against KU-GB-2 antigens was acknowledged specifically with the sera of glioma patients but not with the sera of other cancers and other brain disease patients or healthy persons, KU-GB-2 antigens are considered to be effective as diagnostic drugs or therapy products for glioma. And KU-GB-5 antigens are considered to be effective as serum diagnosis products or therapy products for glioma since they specifically react with the sera of glioma patients. Regarding for the expression in testis, there considered to be little possibility that impaired testis could be a problem when KU-GB-2 antigens or KU-GB-5 antigens are practically used for immunotherapy since testis is a tissue which is insulated from immune system.

### Industrial Applicability

A glioma antigen or a glioma antigen gene DNA obtained by a method for preparing a glioma antigen and/or a glioma antigen gene of the present invention is effective as for therapy and diagnosis of cancers such as glioma and the like, and for obtaining the basic knowledge of sideration of glioma.

1. A method for preparing a glioma antigen and/or a glioma antigen gene comprising the following processes:
(1) A process of constructing λ phage cDNA library comprising the steps of: total RNA is extracted and purified from a glioma cell line; cDNA is synthesized with the purified mRNA, and this cDNA is introduced into λ phage vector to infect E. coli;
(2) A process of generating a reactive serum by eliminating reactants after applying the extract of E. coli to the diluted serum of a glioma patient;
(3) A process of detecting positive clones which are reacted by an antibody in the serum by using a labeled anti-IgG antibody after applying the aforementioned λ phage cDNA library to the reactive serum;
(4) A process of confirming antibody-reactivity after repeating several screenings for the detected positive clones;
(5) A process of a serum screening using the antigens isolated from the positive clones together with at least the sera of glioma patients and healthy persons.

2. A diagnostic drug for detecting glioma comprising an antibody which specifically binds to whole or part of one or more types of glioma antigens obtained by the method for preparing glioma antigens and/or glioma antigen genes as described in claim 1, and/or to whole or part of said glioma antigens.

3. A detecting and diagnostic probe for glioma comprising whole or part of antisense strand of DNA or RNA encoding one or more types of glioma antigens obtained by the method for preparing glioma antigens and/or glioma antigen genes as described in claim 1.

4. An anti-tumor agent with an antibody specifically binding to whole or part of one or more types of glioma antigens obtained by the method for preparing glioma antigens and/or a glioma antigen genes described in claim 1, and/or to whole or part of said glioma antigens as an effective ingredient.

5. DNA encoding the following protein (a) or (b).
(a) A protein comprising the amino acid sequence shown by SEQ ID NO:2;
(b) A protein which comprises an amino acid sequence wherein one or a few amino acids are deleted, substituted or added in the amino acid sequence shown by SEQ ID NO:2, and which has the immunity-inducing activity.

6. DNA comprising the base sequence shown by SEQ ID NO:1 or its complementary sequence and part or whole of these sequences.

7. DNA which hybridizes under a stringent condition with DNA of claim 6, and which encodes a protein having the immunity-inducing activity.

8. DNA encoding the following protein (a) or (b).
(a) A protein comprising the amino acid sequence shown by SEQ ID NO:4, 6, 8, or 10;
(b) A protein which comprises an amino acid sequence wherein one or a few amino acids are deleted, substituted or added in the amino acid sequence shown by SEQ ID NO:4, 6, 8, or 10, and which has the immunity-inducing activity.

9. DNA comprising the base sequence shown by SEQ ID NO:3, 5, 7, or 9, or its complementary sequence and part or whole of these sequences.

10. DNA which hybridizes under a stringent condition with DNA of claim 9, and which encodes a protein having the immunity-inducing activity.

11. DNA encoding the following protein (a) or (b).
(a) A protein comprising the amino acid sequence shown by SEQ ID NO:12;
(b) A protein which comprises an amino acid sequence wherein one or a few amino acids are deleted, substituted or added in the amino acid sequence shown by SEQ ID NO:12, and which has the immunity-inducing activity.

12. DNA comprising the base sequence shown by SEQ ID NO:11 or its complementary sequence and part or whole of these sequences.

13. DNA which hybridizes under a stringent condition with DNA of claim 12, and which encodes a protein having the immunity-inducing activity.

14. A protein comprising the amino acid sequence shown by SEQ ID NO:2.

15. A protein which comprises an amino acid sequence wherein one or a few amino acids are deleted, substituted or added in the amino acid sequence shown by SEQ ID NO:2, and which has the immunity-inducing activity.

16. A protein comprising the amino acid sequence shown by SEQ ID NO:4, 6, 8, or 10.

17. A protein which comprises an amino acid sequence wherein one or a few amino acids are deleted, substituted or added in the amino acid sequence shown by SEQ ID NO:4, 6, 8, or 10, and which has the immunity-inducing activity.

18. A protein comprising the amino acid sequence shown by SEQ ID NO:12.

19. A protein which comprises an amino acid sequence wherein one or a few amino acids are deleted, substituted or added in the amino acid sequence shown by SEQ ID NO:12, and which has the immunity-inducing activity.

20. A peptide comprising a partial protein of any of claims 14 to 19, and which has the immunity-inducing activity.

21. A fusion protein or a fusion peptide wherein the protein of any of claims 14 to 19 or the peptide of claim 20, and a marker protein and/or a peptide tag, are bound.

22. An antibody against the protein of any of claims 14 to 19 or the peptide of claim 20.

23. A host cell comprising an expression system capable of expressing the protein of any of claims 14 to 19 or the peptide of claim 20.

24. A non-human animal whose gene function to encode the protein of any of claims 14 to 19 or the peptide of claim 20 is deficient on its chromosome, or which over-expresses the protein of any of claims 14 to 19 or the peptide of claim 20.

25. A screening method for a promoter or a suppressor for the immunity-inducing activity wherein the immunity-inducing activity in T cell is measured and assessed by using the protein of any of claims 14 to 19 or the peptide of claim 20, a test substance, and T cell.
